(19) United States
(12) Patent Application Publication
(10) Pub. No.: US 2007/0066977 A1
(43) Pub. Date: Mar. 22, 2007

(54) Exchange system for axial spinal procedures

(76) Inventors: Robert L. Assell, Wilmington, NC (US); Stephen D. Ainsworth, Wilmington, NC (US); Andrew H. Cragg, Edina, MN (US); Eugene A. Dickhudt, St. Paul, MN (US); Michael P. Barnhouse, Wilmington, NC (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/501,351

(22) Filed: Aug. 9, 2006

Related U.S. Application Data
(63) Continuation-in-part of application No. 10/972,065, filed on Oct. 22, 2004.
(60) Provisional application No. 60/706,704, filed on Aug. 9, 2005.

(51) Int. Cl. A61B 17/60

(52) U.S. Cl. 606/96

(57) ABSTRACT

An exchange system is disclosed, for providing a protected path to a subcutaneous procedure site. An exchange cannula is provided with a central lumen, and a drill wire lumen that serves as a portal for a drill wire for coupling the assembly to bone. The wall thickness of the exchange cannula may be eccentric, to accommodate the drill wire lumen within the exchange cannula wall. A tensioning handle may be carried over the exchange cannula, for engaging adjacent tissue. The exchange cannula may have a proximal “T” handle. An exchange rod is movably positionable within the central lumen of the exchange cannula.

### CROSS-REFERENCE TO RELATED APPLICATIONS

This U.S. Patent Application claims priority and benefits from U.S. Provisional Patent Application No. 60/706,704, filed on Aug. 9, 2005, and is a continuation-in-part of U.S. patent application Ser. No. 10/972,065, filed on Oct. 22, 2004. The contents of each of the aforementioned U.S. Patent Applications are hereby incorporated in their entirety into this disclosure by reference.

### BACKGROUND OF THE INVENTION

1. Field of the Invention

The present invention relates generally to instrumentation systems and methods for accessing and preparing treatment sites within the spine (e.g., inter-vertebral motion segments) for subsequent therapeutic procedures, such as, for example, spinal arthroplasty, partial or total disc replacement, annulus repair, vertebroplasty, arthrodesis (fusion), or the like. Disclosed herein are various tools and methods of use for performing any number of minimally-invasive treatment procedures (e.g., low trauma disc nucleectomy via trans-sacral axial access).

The invention in particular comprises an exchange system assembly that provides a protected portal through surrounding tissue for insertion of instrumentation, stabilization or other therapeutic devices into or through the sacrum that have outer diameters that are larger that those of the working or docking portals used to create access to a treatment site, e.g., an intervertebral disc space. In general, as disclosed and described below, the exchange system assembly comprises a plurality of component parts, including an exchange rod and an exchange cannula sub-assembly working in combination over and through one another.

2. Description of the Related Art

Chronic lower back pain is a primary cause of lost work days in the United States, and as such is a significant factor affecting both workforce productivity and health care expense. Therapeutic procedures for alleviating back pain range from conservative methods, e.g., with intermittent heat, rest, rehabilitative exercises, and medications to relieve pain, muscle spasm, and inflammation, to progressively more active and invasive surgical means which may be indicated if these treatments are unsuccessful, including various spinal arthroplasties, and eventually even spinal arthrodesis, i.e., surgical fusion.

There are currently over 700,000 surgical procedures performed annually to treat lower back pain in the U.S. In 2004, it is conservatively estimated that there were more than 200,000 lumbar fusions performed in the U.S., and more than 300,000 worldwide, representing approximately a $1B endeavor in an attempt to alleviate patients' pain. In addition, statistics show that only about 70% of these procedures performed may have been successful in achieving this end.

Moreover, there may be multiple causes for a patient's lower back pain, where the pain generators are hypothesized to comprise one or more of the following: bulging of the posterior annulus or PLL with subsequent nerve impingement; tears, fissures or cracks in the outer, innervated layers of the annulus; motion induced leakage of nuclear material through the annulus and subsequent irritation of surrounding tissue in response to the foreign body reaction, or facet pain. Generally it is believed that 75% of cases are associated with degenerative disc disease, where the intervertebral disc of the spine suffers reduced mechanical functionality due to dehydration of the nucleus pulposus.

The intervertebral discs, located anterior to the vertebral canal, are formed of fibrous cartilage, and comprise the posterior and anterior longitudinal ligaments and the annulus fibrosis, circumferentially enclosing a central mass, the. The nucleus pulposus provides for cushioning and dampening of compressive forces to the spinal column. In a healthy adult spine, it comprises 80% water.

Surgical procedures, such as spinal fusion and discectomy, may alleviate pain, but do not restore normal physiological disc function.

With reference to FIGS. 1A and 1B, the vertebrae are the bony building blocks of the spine. Between each of the vertebral bodies are the spinal discs and this unit, comprising two vertebral bodies interfaced by an intermediate spinal disc, is known as a spinal motion segment. The spine has seven vertebrae in the neck (cervical vertebrae), twelve vertebrae in the mid-back (thoracic vertebrae), and five vertebrae in the low back (lumbar vertebrae). All of the vertebrae and discs are held together or surrounded by means of ligaments, which are strong fibrous soft tissues that firmly attach bones to bones. Ligaments contribute to the normal physiologic range of motion of the spine, and if injured, e.g., due to disc degeneration (described below) and ensuing impact on distribution of physiologic loads, they similarly may contribute to the resulting pain.

Thus, the bony spine is designed so that vertebrae “stacked” together can provide a movable support structure while also protecting the spinal cord's nervous tissue that extends down the spinal column from the brain from injury. Each vertebra has a spinous process, which is a bony prominence behind the spinal cord that shields the cord's nerve tissue. The vertebrae also have a strong bony “body” in front of the spinal cord to provide a platform suitable for weight-bearing.

The spinal discs serve as “dampeners” between each vertebral body that minimize the impact of movement on the spinal column. Each disc is comprised of the nucleus pulposus, a central, softer component, contained with in the, a surrounding outer ring.

With age, the water and protein content of the body's cartilage changes resulting in thinner, more fragile cartilage. Hence, the spinal discs and the facet joints that stack the vertebrae, both of which are partly composed of cartilage, are subject to similar degradation over time. The gradual deterioration of the disc between the vertebrae is known as degenerative disc disease, or spondylosis. Spondylosis is depicted on x-ray tests or MRI scans of the spine as a narrowing of the normal “disc space” between adjacent vertebrae.

Radiculopathy refers to nerve irritation caused by damage to the disc between the vertebrae. This occurs because of degeneration of the annulus fibrosis of the disc, or due to traumatic injury, or both. Weakening of the annulus may lead to disc bulging and herniation, i.e., the nucleus pulposus or softer portion of the disc can rupture through the annulus and abut the spinal cord or its nerves as they exit the bony spinal column. When disc herniation occurs, the rupture of the nucleus pulposus the annulus fibrosis may irritate adjacent nervous tissue, causing local pain, or discogenic pain, in the affected area. Any level of the spine can be affected by disc degeneration. When disc degeneration affects the spine of the neck, it is referred to as cervical disc disease, while when the mid-back is affected, the condition is referred to as thoracic disc disease. Disc degeneration that affects the lumbar spine causes pain localized to the low back and is sometimes common in older persons and known as lumbago Degenerative arthritis (osteoarthritis) of the facet joints is also a cause of localized lumbar pain that can be diagnosed via x-ray analysis.

The pain from degenerative disc or joint disease of the spine may be treated conservatively with intermittent heat, rest, rehabilitative exercises, and medications to relieve pain, muscle spasm, and inflammation, but if these treatments are unsuccessful, progressively more active interventions may be indicated, including spinal arthroplasty including prosthetic nucleus device implantation; annulus repair, and total disc replacement, and eventually, even spinal arthrodesis. The intervention performed depends on the overall status of the spine, and the age and health of the patient. Procedures include removal of the herniated disc with laminotomy (a small hole in the bone of the spine surrounding the spinal cord), laminectomy (removal of the bony wall), by needle technique through the skin (percutaneous discectomy), disc-dissolving procedures (chemonucleolysis), and others.

When narrowing of the spaces in the spine results in compression of the nerve roots or spinal cord by bony spurs or soft tissues, such as discs, in the spinal canal this condition is known as spinal stenosis. Spinal stenosis occurs most often in the lumbar spine, i.e., the lower back, but also occurs in the cervical spine and less often in the thoracic spine. It is most often caused by degeneration of the discs between the vertebrae due to osteoarthritis. Rheumatoid arthritis usually affects people at an earlier age than osteoarthritis does and is associated with inflammation and enlargement of the soft tissues of the joints. The portions of the vertebral column with the greatest mobility, i.e., the cervical spine, are often the ones most affected in people with rheumatoid arthritis. Non-arthritic causes of spinal stenosis include tumors of the spine, trauma, Paget's disease of bone, and fluorosis

In the context of the present invention, therapeutic procedures to alleviate pain are restore function are described in a progression of treatment from spinal arthroplasty to spinal arthrodesis. As used herein, spinal arthroplasty encompasses options for treating disc degeneration when arthrodesis is deemed too radical an intervention based on an assessment of the patient's age, degree of disc degeneration, and prognosis.

A wide variety of efforts have been proposed or attempted in the prior art, in an effort to relieve back pain and restore physiological function. Notwithstanding these efforts, there remains a need for methods and tools for accessing and preparing an intervertebral motion segment for subsequent therapeutic procedures, which can be accomplished in a minimally invasive manner.

### SUMMARY OF THE INVENTION

There is provided in accordance with one aspect of the present invention, an exchange system for increasing the cross sectional area of an access pathway through soft tissue to a surface of a bone. The exchange system comprises an elongate tubular exchange bushing, sometimes referred to herein as a rod, having a proximal end, a distal end and a central lumen extending therethrough. An elongate tubular exchange cannula, having a proximal end, a distal end and a central lumen extending therethrough is also provided. The exchange cannula comprises a beveled distal end. A transverse handle is provided on the proximal end of the exchange cannula, and the exchange bushing is axially slidable within the central lumen of the exchange cannula.

The beveled distal end on the exchange cannula generally resides at an angle within the range of from about 20° to about 70° with respect to a longitudinal axis of the exchange cannula. In one embodiment, the angle is about 45°. In another embodiment, the angle is about 30°.

The exchange bushing comprises a tapered transition between a proximal section having a first, greater diameter, and a distal section having a second, smaller diameter. The tapered transition may be inclined at a non-normal angle to the longitudinal axis of the exchange bushing. In one embodiment, the angle of inclination is about 30°. In an alternate embodiment, the angle of inclination is about 45°.

The exchange cannula tube may have a wall thickness that varies from a relatively thick side of the exchange cannula tube to a relatively thin side on an opposing side of the exchange cannula tube. A drill wire lumen may extend axially through the wall of the exchange cannula tube in the thick side.

The exchange system may additionally include a drill wire. In one arrangement, the drill wire is positioned within the lumen or within a hypotube positioned within the lumen.

In accordance with a further aspect of the present invention, there is provided a method of advancing a device, implant or other instrumentation along an axially oriented tract. The method comprises the steps of advancing an exchange rod over a guide pin extending along the tract. An exchange cannula subassembly is advanced over the exchange rod. The exchange cannula subassembly is secured to bone, such as a sacrum. The exchange rod is removed from the tract, and the device implant or instrumentation is introduced along the tract through a central lumen of an exchange cannula tube of the exchange cannula subassembly.

The securing step may comprise securing a drill wire which extends axially through the wall of the exchange cannula tube, into the bone.

Further features and advantages of the present invention will become apparent to those of skill in the art in view of the detailed description of preferred embodiment which follows, when considered together with the attached drawings and claims.

### BRIEF DESCRIPTION OF THE DRAWING

FIG. 1A provides a lateral view of a normal spinal column.

FIG. 1B illustrates examples of normal, degenerated, bulging, herniated, and thinning spinal discs.

FIG. 1C is a lateral view of the lumbar and sacral portion of the spinal column depicting the visualized anterior axial instrumentation/implant line (AAIIL) extending cephalad and axially from the anterior laminectomy site target point.

FIG. 1D is an illustration of an anterior target point on the sacrum

FIGS. 1E and 1F are cross-sectional caudal views of a lumbar vertebrae depicting one and two trans sacral axial implants respectively within corresponding TASII bores formed in parallel with the visualized AAIIL of FIG. 1C.

FIG. 2 is a perspective view of an implantable spinal distraction/fusion rod which is implantable through the exchange systems of the present invention.

FIG. 3 is a side elevational view of the rod of FIG. 2.

FIG. 4 is a side elevational view as in FIG. 3, illustrating different thread relationships of the rod.

FIG. 5 is a side elevated view of an exchange bushing.

FIG. 6 is a side view of one embodiment of an exchange system assembly comprising an exchange bushing and an exchange cannula.

FIG. 7A is a side elevated, cut-away view of one embodiment of an exchange cannula of FIG. 6, in an open configuration.

FIG. 7B is a side elevated view of the exchange cannula of FIG. 6, in a closed configuration.

FIGS. 8A-B illustrate the use of the exchange system of FIGS. 5-7 to deliver a distraction device or an axial spinal implant of larger diameter than a dilator sheath.

FIG. 9A is side cross-sectional view of another embodiment of an exchange system assembly comprising an exchange bushing and an exchange tube.

FIG. 9B is a side cross-sectional view of the exchange bushing of FIG. 9A.

FIG. 9C is a side cross-sectional view of the exchange tube of FIG. 9A.

FIG. 9D is a perspective view of another embodiment of an exchange system comprising an exchange bushing and an exchange tube.

FIG. 9E is a bottom perspective view of the exchange system of FIG. 9D.

FIG. 10A is an exploded, perspective view of components of an exchange system prior to assembly with a drill wire crank handle.

FIG. 10B illustrates components of an exchange system prior to assembly with an alternative drill wire handle.

FIG. 11A illustrates an assembled exchange system with a drill wire crank handle.

FIG. 11B is a proximal end elevational view of an assembled exchange system.

FIGS. 12A and B illustrate a 45 degree exchange cannula tube.

FIG. 13A illustrates an exchange cannula tube with a tract formed in its dorsal surface.

FIG. 13B illustrates an exchange cannula tube with a hypo tube positioned in the tract shown in FIG. 13A.

FIG. 13C illustrates an exchange cannula tube with an integral lumen formed within its dorsal wall.

FIGS. 14A-D illustrate a hypo tube with a distal end angled at 30 degrees.

FIG. 15A is a perspective view and 15B is a side elevational cross section of a tensioning handle on the proximal end of an exchange cannula sub-assembly.

FIG. 16A is a partial cut away view of a coarse-pitch threaded tensioning handle.

FIG. 16B is a front perspective view of a coarse-pitch threaded tensioning handle.

FIG. 17A is a partial cut away view of a fine-pitch threaded tensioning handle.

FIG. 17B is a front perspective view of a fine-pitch threaded tensioning handle.

FIG. 18 is a front perspective view of a shoulder.

FIG. 19A-D illustrates a shoulder affixed at the proximal end of a 30 degree exchange cannula tube.

FIG. 20A illustrates a drill wire positioned within a tract on the dorsal surface of an exchange cannula tube and extending distally past the distal end of the tube.

FIG. 20B illustrates the drill wire and exchange tube of FIG. 20A with a fusion implant extending partially past the distal end of the tube.

FIG. 21A-D illustrate the distal end of a fluted drill wire and dimensions of one embodiment of a fluted drill wire.

FIG. 22A also illustrates a fluted drill wire.

FIG. 22B illustrates a threaded drill wire.

FIG. 22C illustrates a drill wire with a trocar tip.

FIG. 22D illustrates a thumb wheel handle at the proximal end of a drill wire.

FIG. 22E illustrates a crank handle at the proximal end of a drill wire.

FIG. 22F is an end perspective view of an alternate drill wire configuration, having a single “gashed” flute.

FIG. 23A-E illustrate the insert component of a two-part exchange rod.

FIG. 23F illustrates an assembled two-part exchange rod comprising an insert and sheath.

FIG. 24A is a dorsal view of a one-part exchange rod.

FIG. 24B is a ventral view of a one-part exchange rod.

FIG. 24C is a side elevational view of the distal end of a one-part exchange rod.

FIG. 24D is a top plan view of the proximal end of a one-part exchange rod.

FIG. 25 is a perspective view of an eccentric exchange cannula with a proximal “T” handle.

### DETAILED DESCRIPTION OF THE PREFERRED EMBODIMENTS

In accordance with one aspect of the embodiments described herein, there are provided surgical instrumentation systems and techniques for efficiently and atraumatically accessing and preparing treatment sites within the spine, such as, for example, vertebral motion segments, for subsequent therapeutic spinal procedures. In one approach, the step of accessing the treatment site includes using fluoroscopic imaging to visually align one or more components of the instrumentation system via a percutaneous, anterior trans-sacral axial approach. In another aspect, the treatment site includes a spinal disc and the subsequent therapeutic procedure includes nucleectomy. In yet another aspect, the therapeutic procedure includes immobilization devices to facilitate fusion; deployment of augmentation media; deployment of dynamic stabilization implants, e.g., motion preservation devices that preserve or restore physiologic function.

In accordance with one aspect of the embodiments described herein, there are provided surgical tool sets and methods of using the tool sets. The tools of the tools sets can be used individually and/or in combination with each other. As will be explained in further detail below, in one approach, certain tools fit over other tools, and therefore can be used over each other. In another approach, the tools fit through each other, and therefore can be used through one another.

It will be understood that the access methods described can include the step of utilizing an anterior or posterior trans-sacral pathways. The therapies to the spinal discs and vertebral bodies described herein can be conducted on one or more spinal discs or vertebral bodies, or one or more vertebral motion segments. In one approach, therapeutic procedures are performed through or on at least one spinal disc and at least one vertebral body traversed by at least one working channel.

For convenience, the exemplary access by a single anterior method, and treatment of only a single spinal disc or vertebral body is described herein. It will be understood, however, that the tools and methodologies described herein are applicable to any spinal access pathway, including without limitation open surgical procedures from any access orientation, and to any number of spinal discs and/or vertebral bodies.

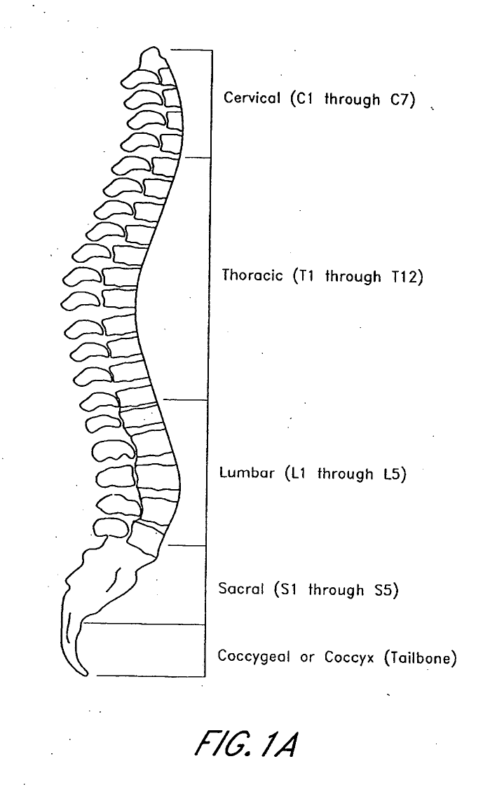

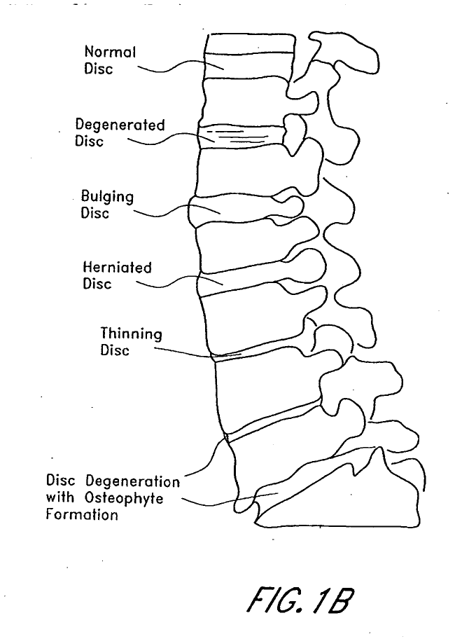

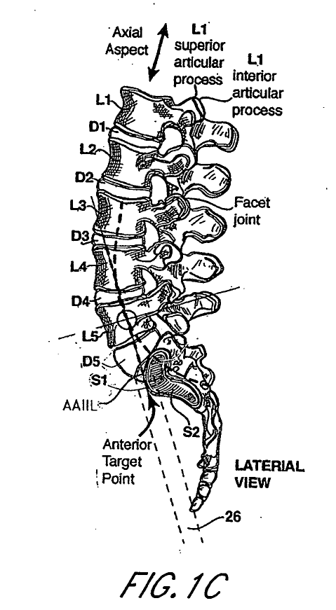

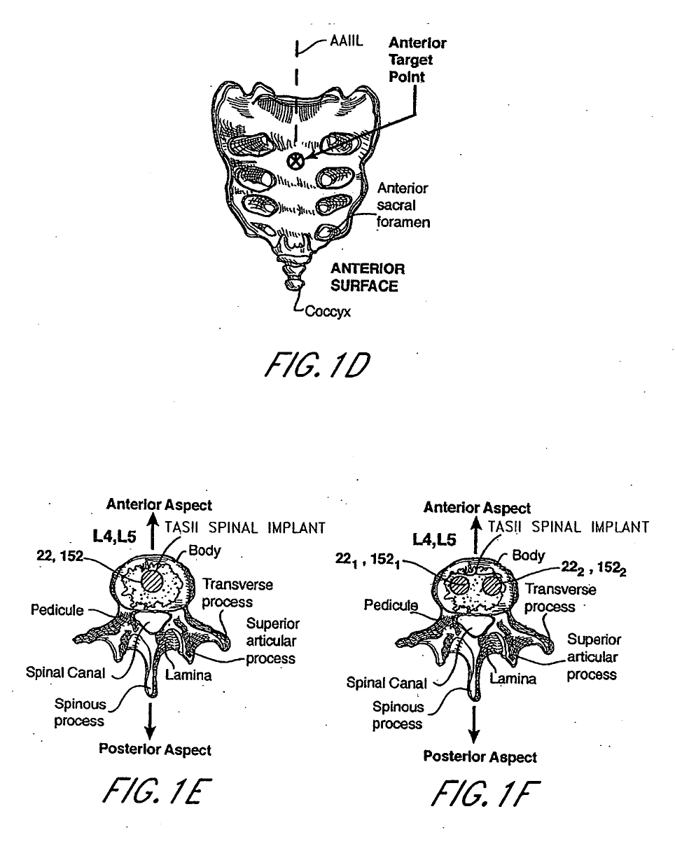

FIGS. 1C-D schematically illustrate the anterior trans-sacral axial spinal instrumentation/implant (TASII) approaches in relation to the lumbar region of the spinal column, and FIGS. 1E-F illustrate the location of a TASII implant or pair of implants within an anterior TASII axial bore 152 or pair of TASII axial bores 22₁, 22₂, or 152₁, 152₂. Two TASII axial bores and spinal implants or rods are shown in FIG. IF to illustrate that a plurality, that is two or more, of the same may be formed and/or employed in side by side relation parallel with the anterior axial instrumentation/implant line (AAIIL).

The lower regions of the spinal column comprising the coccyx, fused sacral vertebrae S1-S5 forming the sacrum, and the lumbar vertebrae L1-L5 described above are depicted in a lateral view in FIG. 1C. The series of adjacent vertebrae located within the human lumbar and sacral spine have an anterior aspect, a posterior aspect and an axial aspect, and the lumbar vertebrae are separated by intact or damaged spinal discs labeled D1-D5 in FIG. 1C. FIG. 1D depicts the anterior view of the sacrum and coccyx.

The method and apparatus for forming an anterior TASII axial bore initially involves accessing an anterior sacral position, e.g. an anterior target point at about the junction of S1 and S2 depicted in FIGS. 1C and 1D. One (or more) visualized, imaginary, axial instrumentation/implant line extends cephalad and axially in the axial aspect through the series of adjacent vertebral bodies to be fused or otherwise treated, L4 and L5 in this illustrated example. The visualized AAIIL through L4, D4, L5 and D5 extends relatively straight from the anterior target point along S1 depicted in FIGS. 1C and 1D, but may be curved as to follow the curvature of the spinal column in the cephalad direction.

It will be noted that the terms trans-sacral axial spinal instrumentation/implant (TASII), and anterior axial instrumentation/implant line (AAIIL), as used herein, are analogous to the terms trans-sacral axial spinal instrumentation/fusion (TASIF), and anterior axial instrumentation/fusion line (AAIFL), The analogous terms generally refer to the same percutaneous pathways, the primary difference being the types of treatments and implants delivered through the respective percutaneous pathways.

U.S. Pat. No. 6,575,979, issued Jun. 10, 2003, titled Method And Apparatus For Providing Posterior Or Anterior Trans-Sacral Access To Spinal Vertebrae, hereby incorporated in its entirety into this disclosure by reference, discloses in detail tools and methodology for accessing targeted treatment sites, such as, for example, inter-vertebral motion segments, and establishing access pathways which can be used by the exchange systems of the present invention.

Certain of the access and preparation surgical tools, as explained in U.S. patent application Ser. No. 10/972,065, filed Oct. 22, 2004, hereby incorporated in its entirety herein by reference, take the form of elongated solid body members extending from proximal to distal ends thereof. Certain cutter tools useful in the context of the present invention are disclosed in U.S. Provisional Patent Application Ser. No. 60/778,035, filed Feb. 28, 2006, the disclosure of which is hereby incorporated in its entirety herein by reference.

Elongated solid body members in medical terminology include, for example, relatively stiff or flexible needles of small diameter typically used to penetrate tissue, wire stylets typically used within electrical medical leads or catheters to straighten, stiffen, or impart a curved shape to the catheter, guidewires that are used to traverse body vessel lumens and access remote points therein (certain hollow body guidewires have lumens for a number of uses), and obturators. Obturators are typically formed as rods provided in various diameters with blunt distal tips that can be manipulated to penetrate, separate or manipulate surrounding tissue without cutting or damaging the tissue.

In accordance with one aspect of the embodiments described herein, there are provided exchange systems providing a protected portal to the target site (e.g., the sacrum) through which instrumentation or implants having O.D. dimensions (e.g., greater than about 0.35″) that are too large to be accommodated through the working and docking portal provided by a large dilator sheath, are inserted through the exchange system, to and through the target site and to the treatment site

For example, the exchange systems described below may be used for implantation of a variety of spinal distraction/fusion rods with varied thread pitch and diameters along different portions of their length, and capable of distracting two or more vertebral bodies relative to each other and/or facilitating the procedure of fusing the vertebral bodies together from within the spine. The exchange systems described below may also be used for implantation and deployment of various prosthetic nucleus or prosthetic disc replacement devices (not shown) that achieve dynamic stabilization of the spine.

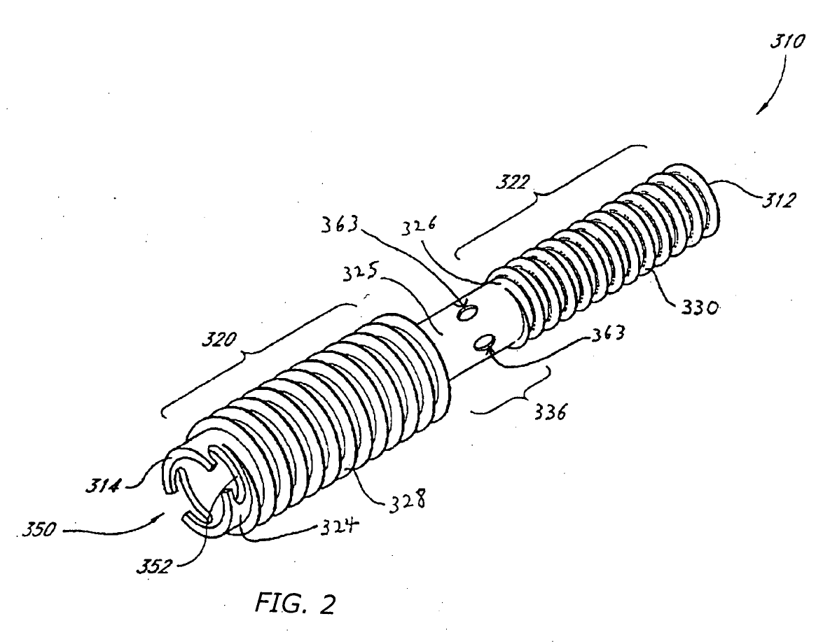

One example of a distraction/fusion rod implantable through the exchange systems disclosed herein is illustrated in FIGS. 2, 3 and 4. The rod 310 extends between a distal, leading end 312 and a proximal, trailing end 314 and comprises a proximal threaded section 320, a distal threaded section 322, and an intermediate section 336 that can be threaded or unthreaded. In one preferred embodiment, bony fusion is bone-to-bone rather than being through the inner diameter of the distraction/fusion rod 310, in contrast to spinal cages known in the art. The rod 310 is typically on the order of 1.25 to 2.25 inches in length for a two vertebral body application. Each of the threaded sections are typically on the order of 0.5 to 1.25 inches in length, whereas the intermediate section, if present, is typically on the order of 0.25 to 0.5 inch in length. It should be noted that the actual dimensions of the rod 310 can vary depending on the physical size and anatomical characteristics of the patient being treated. The rod 310 is typically produced from a biocompatible material, such as, for example, titanium alloy, stainless steel, Nitinol, or various known high strength polymers

The threaded sections 320, 322 of the rod 310 comprise coaxial cylindrical root portions 324, 326 and screw threads 328, 330. The intermediate section 336 comprises a coaxial cylindrical root portion 325 that may be continuous with root portion 326 and that may have the same outer diameter as root portion 326. In one embodiment, illustrated in FIGS. 2-4, the intermediate section 336 has a plurality of side apertures 363 that are in communication with the central lumen 368 of the rod 310. In another embodiment (not shown), the intermediate section 336 is threaded. In yet another embodiment (not shown), the root portion 325 of intermediate section 336 has a diameter that is different from that of root portion 326 of the distal section 322.

The root portions typically have outer diameters that are between approximately 6 mm and approximately 13 mm. With continued reference to the embodiment illustrated in FIG. 4, the outer diameter 342 of the root portion 324 in the proximal section 320 is greater than the outer diameters 344, 346 of the root portions 326, 325 in the distal and intermediate sections 322, 336. In one implementation of the invention, the outer diameter 342 of the root portion 324 in the proximal section 320 is approximately 9-10 mm which is larger than the outer diameters 344, 346 of the root portions 326, 325 in the distal and intermediate sections 322, 336. The outer diameters 344, 346 may be approximately 6 mm in the present implementation. The diameters 342, 344, 346, as illustrated in FIG. 4, are constant along any of the three primary sections 320, 322, 336. In another embodiment (not shown), the outer diameter of the root portion can taper toward the leading end of the one or more of the primary sections 320, 322, 336.

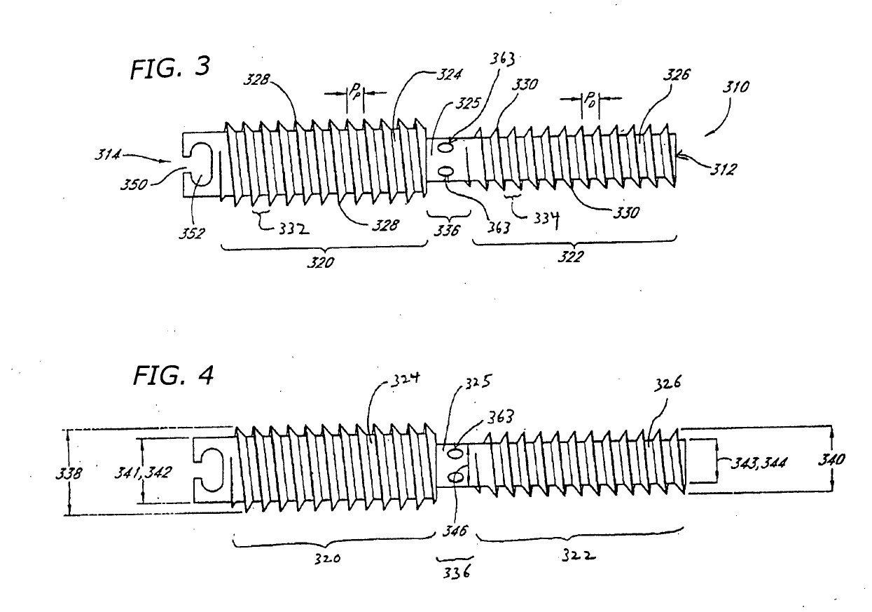

With continued reference to the embodiment shown in FIG. 3, screw threads 328, 330 are formed on root portions 324, 326 and extend as continuous threads from the trailing end to the leading end of the respective threaded sections 320, 322. The screw threads 328, 330 include multiple revolutions that are spaced apart along the roots 324, 326 by interthread spacings 332, 334. The proximal and distal screw threads 328, 330 are like-handed (i.e. the threads turn in the same direction) so that both screw threads are right-handed or so that both are left-handed. In the embodiment illustrated in FIGS. 3 and 4, the screw threads 328, 330 are right-handed.

The screw threads 328, 330 are typical of “cancellous” type bone threads known in the art. The threads 328, 330 are typically cut with generally flat faces on the flights of the thread with the most flat of the faces oriented in the direction of the applied load. The threads 328, 330 are typically self-tapping screws. In one embodiment, the thread profile generally comprises deep flights with an asymmetric thread form, which provides the advantage of improved weight bearing and load distribution. The screw threads 328, 330 have both a major diameter 338, 340 and a minor diameter 341, 343. The minor diameters 341, 343 of the illustrated screw threads 328, 330 are the same as the outer diameters 342, 344 of root portions 324, 326. In another embodiment, the minor diameters 341, 343 are greater than the outer diameters 342, 344. In yet another embodiment, the minor diameters 341, 343 are less than the outer diameters 342, 344. The major and minor diameters within any of the primary sections 320, 322, 336 may be constant throughout the section. In another embodiment, the major and/or the minor diameters of the threads taper from larger to smaller toward the leading end of one or more of the primary sections 320, 322, 336.

With continued reference to the embodiment shown in FIG. 4, the major diameter 338 of the proximal section 320 is greater than the major diameter 340 of the distal section 322. Similarly, the minor diameter 341 of the proximal section 320 is greater than the minor diameter 343 of the distal section 322. The proximal section 320 typically has a major diameter 338 within the range of from about 10 mm to about 15 mm or greater, and often of approximately 12-13 mm, and a minor diameter 341 of approximately 9-10 mm. The distal section 322 typically has a major diameter 340 of approximately 9 mm and a minor diameter 343 of approximately 6 mm. Alternatively, the major diameter 340 may be larger than the minor diameter 341. Additional details of such spinal implants are disclosed in U.S. Pat. No. 6,921,403 to Cragg, et al., titled Method and Apparatus for Spinal Distraction and Fusion, the entirety of which is hereby incorporated by reference herein.

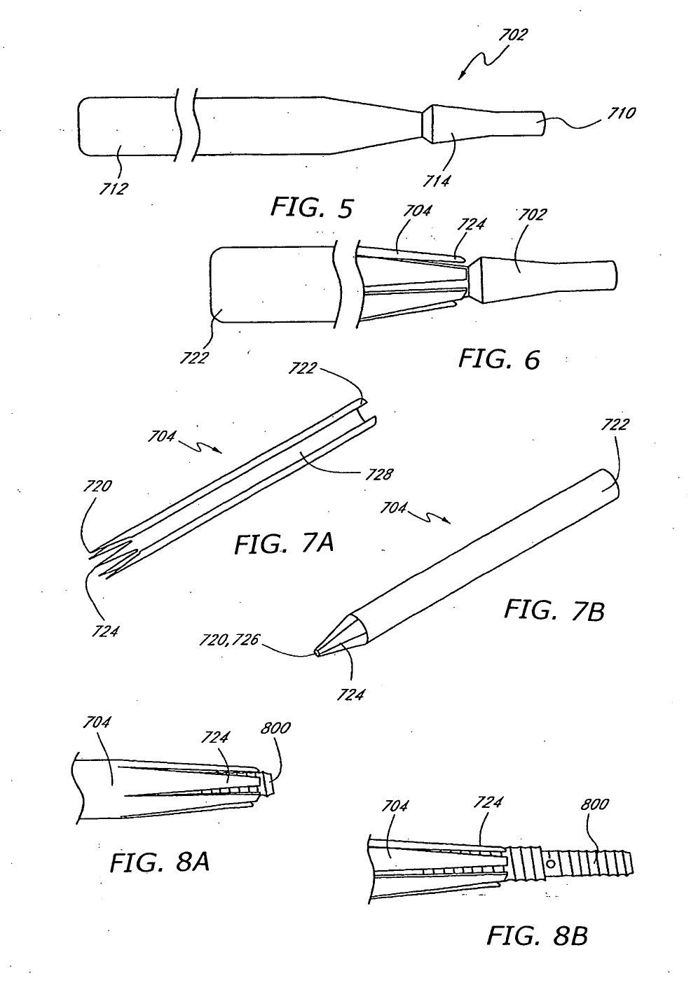

With reference to FIGS. 5-6 and 7A-B, in one embodiment, the exchange system assembly comprises an exchange bushing 702 and an exchange cannula 704.

The shaped exchange bushing 702 extends between a distal end 710 and a proximal end 712. The elongate, cannulated exchange bushing 702 is shaped and tapered toward its distal end 710. In one embodiment, the bushing 702 is cannulated with a central lumen having an inner diameter of about 0.14″ (i.e., slightly larger than a diameter of a typical guide pin). In one embodiment, the length of the bushing 702 is approximately 14.00″.

Bushing 702 has a tapered tip 714 at its distal end 710. In one embodiment, the tapered tip 714 starts at the inner diameter of the bushing 702 and continues at approximately an 18 degree angle for about 0.5″ after which the taper cuts sharply back (i.e., flares out) towards the center of the bushing 702 and begins the taper again at about an 18 degree angle out to the outer diameter of the bushing 702. This creates an annular recess region in which the exchange fingers 724 of the cannula 704 can nest, thereby providing a protected profile during delivery (i.e., the bushing 702 protects the exchange fingers 724) See FIG. 6. Delivery may be accomplished over an extended guide pin.

In one embodiment, the exchange bushing 702 comprises a polymeric material, such as an acetyl copolymer or the like. In another embodiment the exchange bushing 702 is fabricated from a metal or metal alloy, e.g., stainless steel. The exchange bushing 702 can be either machined or injection molded.

As will be described in greater detail below, the exchange bushing may be fabricated or otherwise formed from either metal or polymer, or, alternatively, as a polymeric overmold on a preformed metal bushing subcomponent. The addition of an over molded polymeric tip to produce a polymer-metal hybrid bushing, may provide manufacturing advantages in that producing the distal curvature and or taper of the exchange bushing (rod) in this manner may be more cost effective and provide more design flexibility than machining the entire component from metal. More specifically, to reduce manufacturing complexity and costs, a mold may be developed to mold the polymeric tapered and beveled tip, which would be over molded onto a pre-existing stainless piece of bushing stock. This hybrid configuration would be an alternative to an all stainless or all polymeric construct. Any of a variety of medical grade polymers may be used for the overmolded tip, such as, for example, polysulphones, polyphenylsulphones, PEEK, and others known in the art.

With reference to the embodiments in FIGS. 6 and 7A-B, there is provided an exchange system that comprises a “fingered” exchange cannula 704, which works in combination with the bushing 702. The exchange cannula 704 extends between a distal end 720 and a proximal end 722 and defines an inner lumen 728.

The exchange cannula 704 comprises a plurality of distally extending “fingers” 724 at the distal end 720 that are generally triangular in shape. FIG. 7A shows the exchange cannula 704 in the “open” position with its fingers 724 extended radially outward compared to the “closed” position. FIG. 7B shows the exchange cannula 704 in the “closed” or insertion position with its fingers 724 congregated about a central axis, thereby forming a conical tip 726. The conical tip 726 is designed to enter the sacral bore, and to hold dilation and position intact during subsequent deployment of instrumentation or implants. As will be described in more detail below, in other embodiments a drill wire with various tip configurations can be used to hold dilation and position intact during subsequent deployment of instrumentation or implants.

In one embodiment, the exchange cannula 704 is formed from polymeric tubing (e.g., such as acetal copolymer) In one embodiment, the cannula 704 is about 8.00″ in length, and comprises from 3 to 8 “fingers” 724 at the distal end 720 that are approximately triangular in shape. Here, the fingers 724 are approximately 1.00″ in length and configured so as to collapse towards the longitudinal axis of the cannula at approximately a 30 degree angle.

In one mode of use, the exchange cannula 704 is seated on the outside of the shaped exchange bushing 702 during insertion into the sacrum following removal of the large dilator sheath 220 (i.e., working cannula that was used for cutting and extraction). Once the shaped exchange bushing 702 is seated in the sacrum, the exchange cannula 704 is advanced distally and into place. The fingers 724 of the exchange cannula 704 slip into the hole or entry point leading to the treatment site, and the shaped exchange bushing 702 is withdrawn enabling the insertion of subsequent instrumentation or other devices and implants through the lumen 728 of the exchange cannula 704 and into the treatment site. In one approach, the subsequent instruments can optionally be advanced through the cannula 704 in combination with a guide pin.

With reference to FIGS. 8A-B, the largest O.D. of the to-be-deployed device 800 (i.e., the O.D. toward the proximal end of the device 800) exceeds that of a dilator sheath (not shown but described in co-pending and commonly assigned U.S. patent application Ser. No. 10/972,299 filed Oct. 2, 2004, the entire contents of which are hereby expressly incorporated by reference into this disclosure) and that of the exchange cannula 704 while in its “closed” configuration. The device 800 is subsequently delivered to the treatment site by radially outwardly displacing the fingers 724 of the exchange cannula 704 to create a pathway that has a diameter large enough to accommodate the passage of the device 800, while isolating the working channel from adjacent organs or anatomical structures.

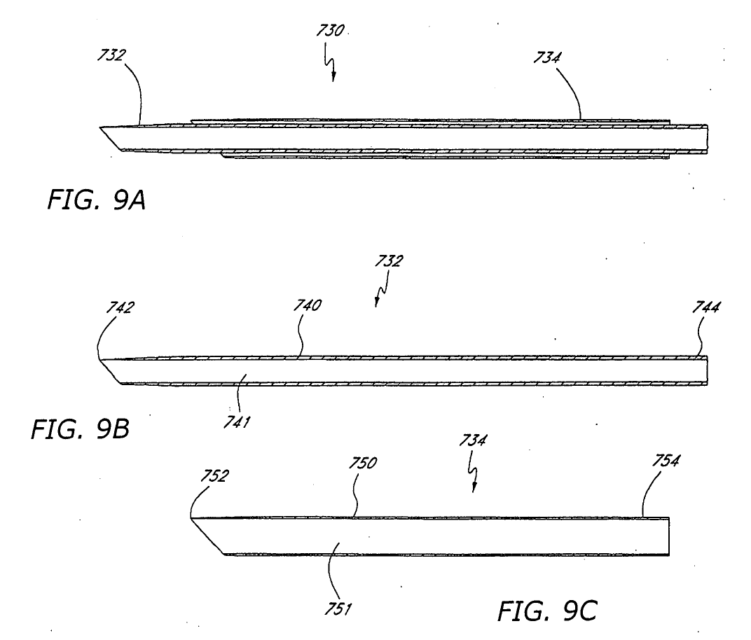

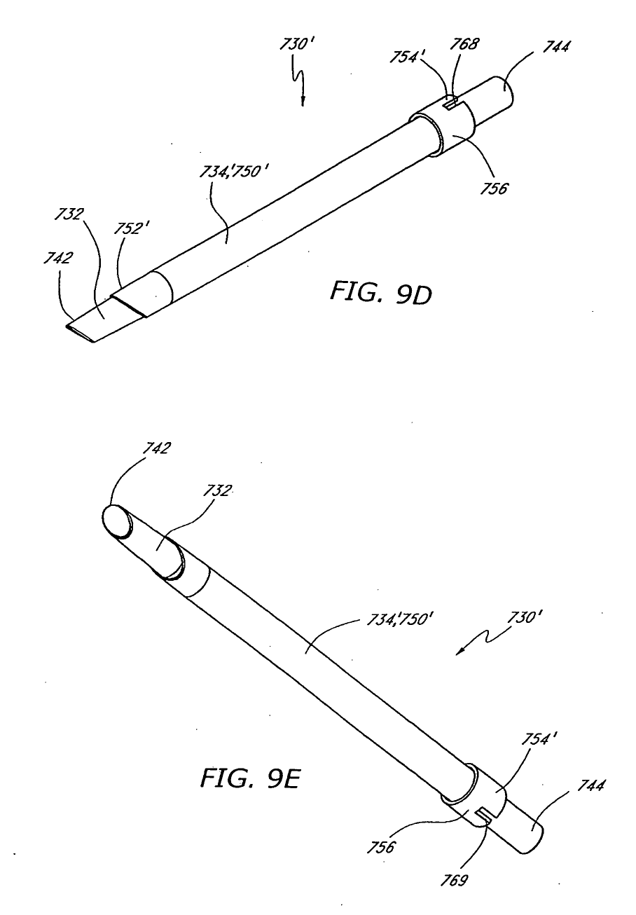

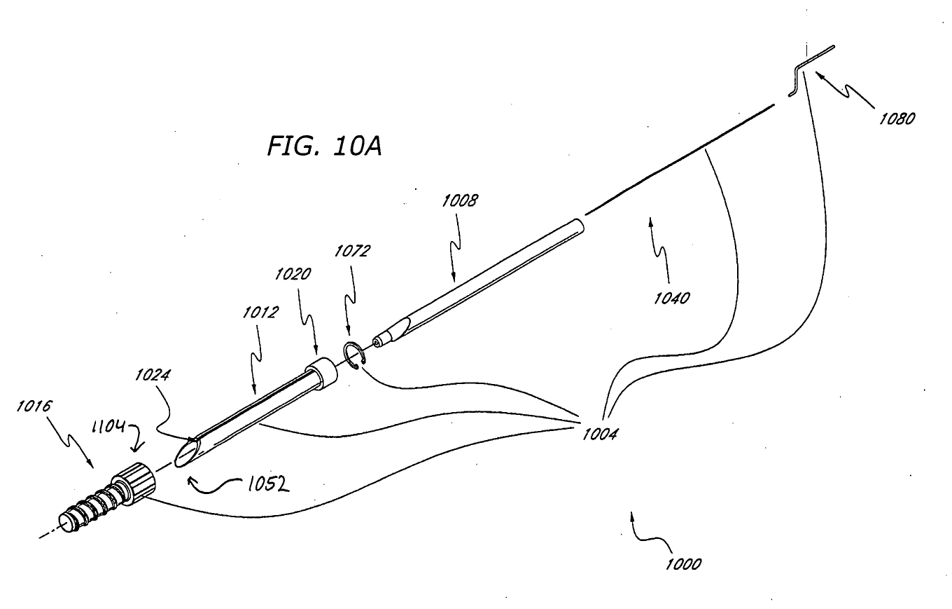

In accordance with another aspect of the embodiments described herein, there is provided an exchange system that provides a protected a portal to a treatment site, and that comprises an exchange bushing and an exchange tube. With reference to FIGS. 9A-C, in one embodiment, there is provided exchange system assembly 730 comprising an exchange bushing 732 and an exchange cannula 734.

The exchange bushing 732 comprises a tube 740 that extends between a distal end 742 and a proximal end 744, and defines an inner lumen 741. The bushing distal end 742 is typically beveled at an angle of about 20° to about 70°, often about 30° to about 60°. In one embodiment, the distal end is beveled at an angle of about 45°. The outside diameter may also be tapered to a reduced diameter at the distal end 742 to facilitate advance through the tissue tract.

The bushing 732 is typically machined from stainless steel, or formed, e.g., by extrusion or injection molding from a polysulphone, or a polymer such as PEEK or any other known suitable material.

The exchange cannula 734 comprises a tube 750 that extends between a distal end 752 and a proximal end 754, and defining an inner lumen 751. The tube distal end 752 is typically beveled at an angle of about 20° to about 70°, often about 30° to about 60°. In one embodiment, the distal end 752 is beveled at an angle of about 45°.

The exchange cannula 734 is typically formed from stainless steel, or from a suitable polymer, such as polysulfones, polypheylsulfones, acetal copolymer, or the like.

With reference to the exchange assembly 730 shown in FIG. 9A, the distal portion of the exchange bushing 732 protrudes from distal end 752 of the exchange tube 734. In one mode of use, the bushing 732 is distally advanced into the sacrum over a dilator sheath (not shown). Once the bushing 732 is advanced over the sheath and seated on the sacrum, the exchange cannula 734 is distally advanced over the bushing 732 and into place. The bushing 732 is then withdrawn over the dilator sheath, which is then also removed, enabling the insertion of subsequent instruments, devices, or implants through the lumen 751 of the tube 734. In one embodiment, the subsequent instruments, devices, or implants are advanced through the lumen 751 over a guidewire. In another embodiment, the subsequent instruments, devices, or implants are advanced through the lumen 751 without the aid of a guidewire.

With reference to FIGS. 9D-E, in yet another embodiment, the exchange system 730′ comprises a bushing 732 and an exchange cannula 734′. The exchange cannula 734′ comprises a handle such as an annular band 756 at the proximal end 754′. The annular band 756 or other aspect of proximal end 744 comprises one or more indicium such as lines, pins or notches 768, 769, as orientation indicators to show the rotational alignment of the bevel of the distal end 752′ of the exchange cannula 734′.

In accordance with a further aspect of the present invention, there is provided an alternative exchange system for providing a protected portal to a treatment site, for example, a patient's sacrum, for the insertion of instrumentation or implants having outer diameters that are too large to be accommodated through the working and docking portal provided by a large dilator sheath, such as the working cannula previously disclosed in the co-pending and commonly assigned applications noted above. In some embodiments, the instruments or implants to be inserted have outer diameters greater than about 0.35″. One specific advantage over current practice offered by the exchange system embodiments of the present invention and described below is their “hands-free” operability. In one embodiment, the exchange system of the present invention provides for “de-tensioning” of the spring-like resistance of surrounding soft tissue during advancement of the exchange cannula, and results in an exchange system that is effectively supported by and held in place via compression against the sacrum. In yet another aspect of the present invention, there is provided a drill wire which, when tapped or torqued into the sacrum, serves to secure the exchange cannula sub-assembly by such anchoring into the sacrum, to facilitate deployment of implants to and/or through the target site for therapy at a treatment site. These and other advantages and features of the exchange system embodiments and techniques disclosed in the present invention will be more readily understood from the following detailed description and figures.

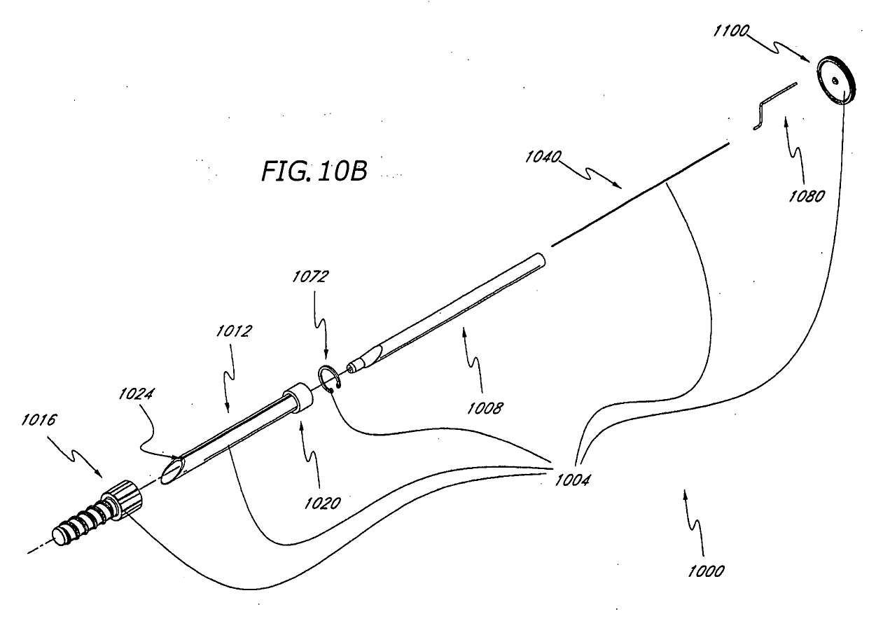

Aspects of the present exchange system 1000 are depicted in FIGS. 10A-10B for providing an axially oriented access tract. Exchange system 1000 comprises an exchange cannula sub-assembly 1004 and an exchange bushing or rod 1008. Any of the exchange system components disclosed herein may be provided with a coating such as a surfactant, hydrogel or other hydrophilic coating to facilitate movement between adjacent components.

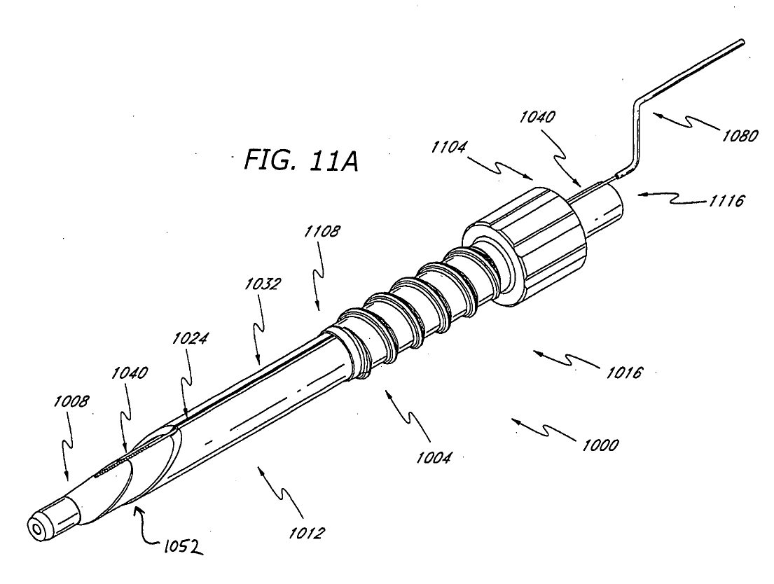

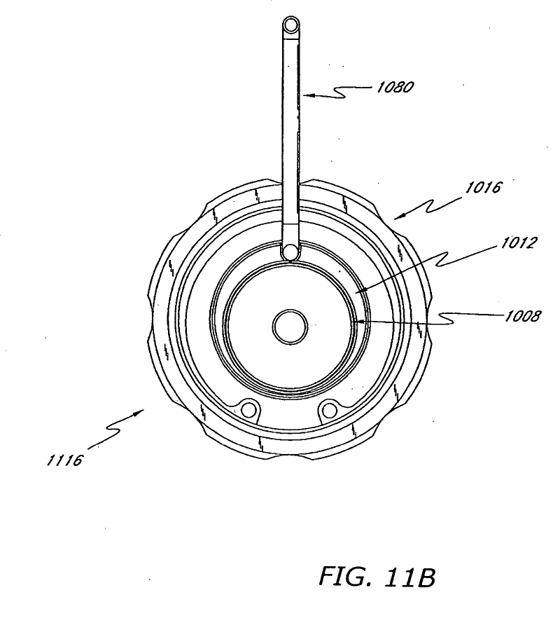

In preferred embodiments, exchange cannula sub-assembly 1004 comprises an exchange cannula tube 1012 with a drill wire portal 1024, a tensioning handle 1016, a shoulder 1020, retaining means 1072, a drill wire 1040, and a drill wire handle 1100 or crank handle 1080. FIGS. 10A-10B show these component parts of exchange cannula sub-assembly 1004 and exchange rod 1008 of exchange system 1000. During assembly, the proximal end 1104 of tensioning handle 1016 is passed over the distal end 1052 of exchange cannula tube 1012 until proximal end 1104 sits over shoulder 1020. An assembled exchange system 1000 with a drill wire crank handle 1080 is shown in FIG. 11A. FIG. 11B shows a posterior view (proximal end elevation) of the proximal end 1116 of the assembled exchange system 1000. Crank handle 1080, tensioning handle 1016, exchange cannula tube 1012, and exchange rod 1008 can be seen in the posterior view of FIG. 11B.

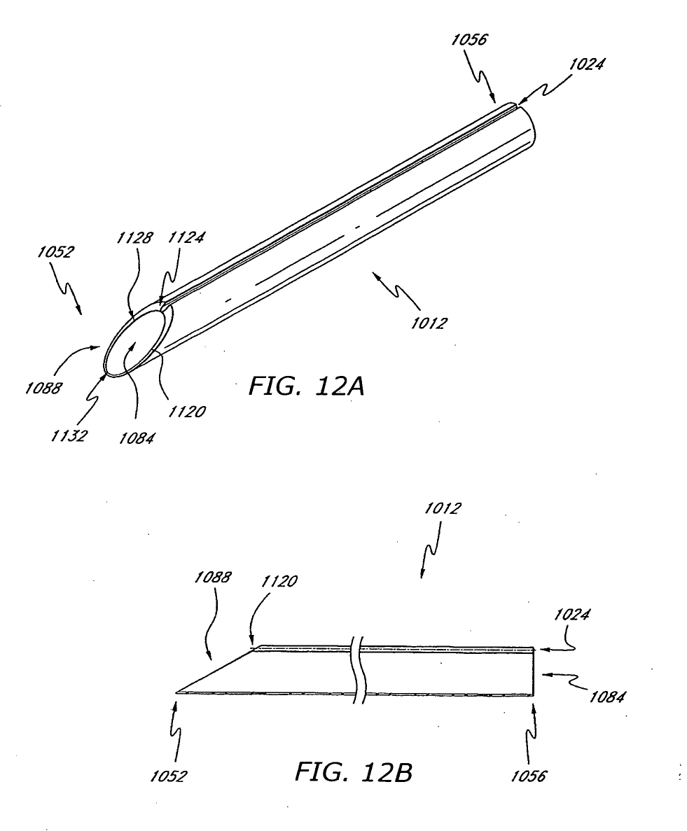

As shown in FIGS. 12A-12B, exchange cannula tube 1012 comprises a proximal end 1056, a distal end 1052, and a central lumen 1084. Distal end 1052 comprises a bevel 1088 at its distal end. Bevel 1088 comprises at least one surface 1120 slanted relative to a longitudinal axis of exchange cannula tube 1012 at an angle ranging from about 20 to about 70 degrees. In some embodiments, bevel 1088 comprises a surface 1120 slanted relative to a longitudinal axis of exchange cannula tube 1012 at an angle ranging from 30 to 60 degrees. In preferred embodiments, bevel 1088 comprises a surface 1120 slanted relative to a longitudinal axis of exchange cannula tube 1012 at an angle often of about 30 degrees (30 degree exchange cannula tube) or often of about degrees (45 degree exchange cannula tube); the latter is illustrated in the side view of exchange cannula tube 1012 in FIG. 12B.

Exchange cannula tube 1012 may be elongated and eccentric in terms of wall thickness as illustrated in FIG. 12A. The thickest portion 1124 of the wall 1128 of tube 1012 can have from about 2 to about 6 times the thickness of the thinnest portion 1132 of the wall 1128. For example, the thickest portion 1124 can range from about 0.280″ (7 mm) to about 0.360″ (9 mm) and the thinnest portion 1132 can range from about 0.060″ (1.5 mm) to about 0.145″ (3 mm). In some embodiments, exchange cannula tube 1012 is concentric in terms of wall thickness. For example, the thickness of wall 1128 can range from about 0.025″ (0.5 mm) to about 0.213″ (5.5 mm). Exchange cannula tube 1012 has a length ranging from about 6.00″ (150 mm) to about 10.00″ (255 mm). In a preferred embodiment, exchange cannula tube 1012 has a length of about 8.00″ (200 mm). The diameter of central lumen 1084 can range from about 0.375″ (9 mm) to about 0.650″ (17 mm) In preferred embodiments the diameter of central lumen 1084 measures about 0.560″ (14 mm). The outer diameter of exchange cannula tube 1012 can range from about 0.650″ (17 mm) to about 0.800″ (20 mm) and in preferred embodiments is about 0.685″ (18 mm).

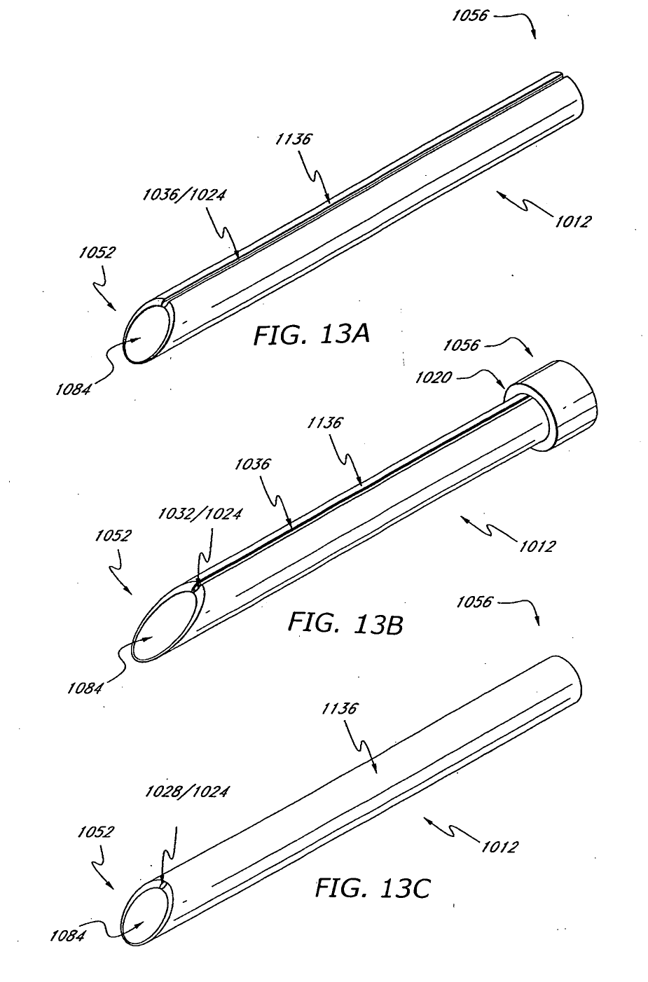

In some aspects of the present invention, as shown in FIGS. 13A-13C, exchange cannula tube 1012 is configured to comprise a drill wire lumen or portal 1024 in its dorsal surface 1136 for passage of drill wire 1040. In the context of the present invention, as used herein dorsal surface refers to the top surface of the instrument, when viewed from the perspective of the clinician, in use. In one embodiment, dorsal surface 1136 of the wall of exchange cannula tube 1012 is milled or otherwise formed with a groove or tract 1036 that provides a lumen 1024 and extends in parallel to the longitudinal axis of exchange cannula tube 1012, as shown in FIG. 13A. FIG. 13B shows exchange cannula tube 1012 with shoulder 1020 affixed at proximal end 1056 and with a hypo tube 1032 disposed within, affixed and/or secured in tract 1036 and serving as drill wire portal 1024. In another embodiment, portal 1024 is formed by molding or extrusion, as an integral lumen 1028 along the longitudinal axis of dorsal surface 1136 of exchange cannula tube 1012, as shown in FIG. 13C. In an alternative embodiment (not shown), hypo tube 1032 may be seated along the inside or outside of dorsal surface 1136 of an ungrooved/unmilled exchange cannula tube 1012, rather than recessed within tract 1036, although this configuration is generally less preferred as it decreases the inside diameter or increases the outer diameter of exchange system assembly 1000 and hence the resulting reduction in useful ID or increased dilation of tissue caused during deployment.

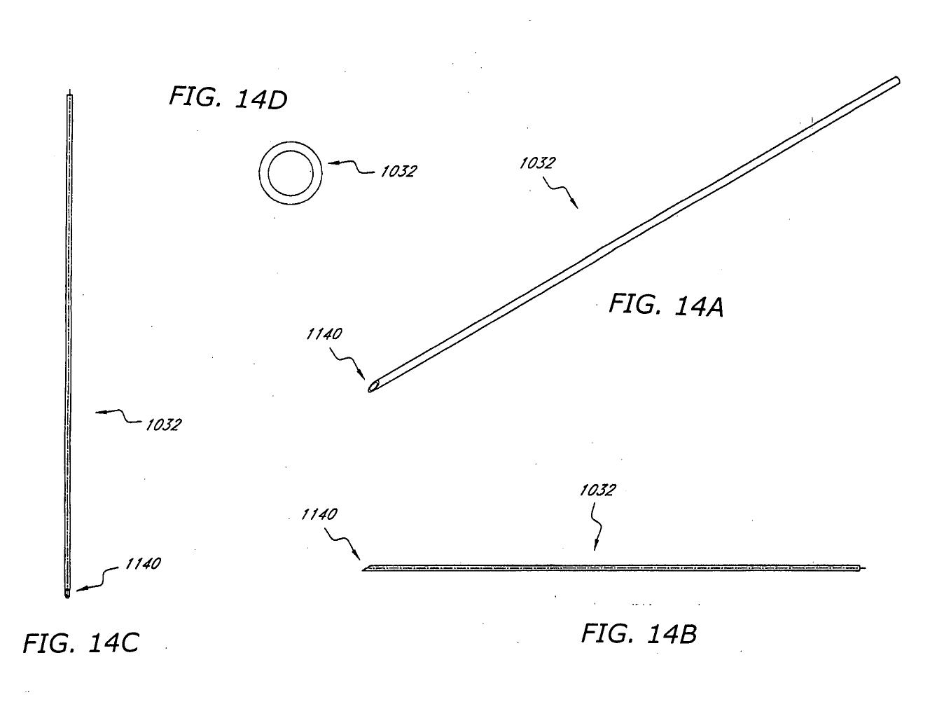

As shown in FIGS. 14A-14D, one embodiment of hypo tube 1032 comprises a distal end 1140 angled at 30 degrees with respect to a longitudinal axis of an exchange cannula tube 1012 for use with a 30 degree exchange cannula tube 1012. Distal end 1140 can also be angled at other angles, including 45 degrees, for use with different embodiments of exchange cannula tube 1012. Hypo tube 1032 can be constructed from metal or metal alloy, such as 300 series stainless steel, and is generally about the same length as dorsal surface 1136 of exchange cannula tube 1012. In some preferred embodiments, hypo tube 1032 is about 7.00″ long (175 mm). The inner diameter of hypo tube 1032 can range from about 0.030″ (0.7 mm) to about 0.070″ (1.8 mm) and is often about 0.050″ (1.3 mm). The outer diameter of hypo tube 1032 ranges from about 0.050″ (1.3 mm) to about 0.090″ (2.3 mm) and is often about 0.070″ (1.8 mm). The inner and outer diameters of hypo tube 1032 can be seen in FIG. 14D which is a cross-sectional view of hypo tube 1032.

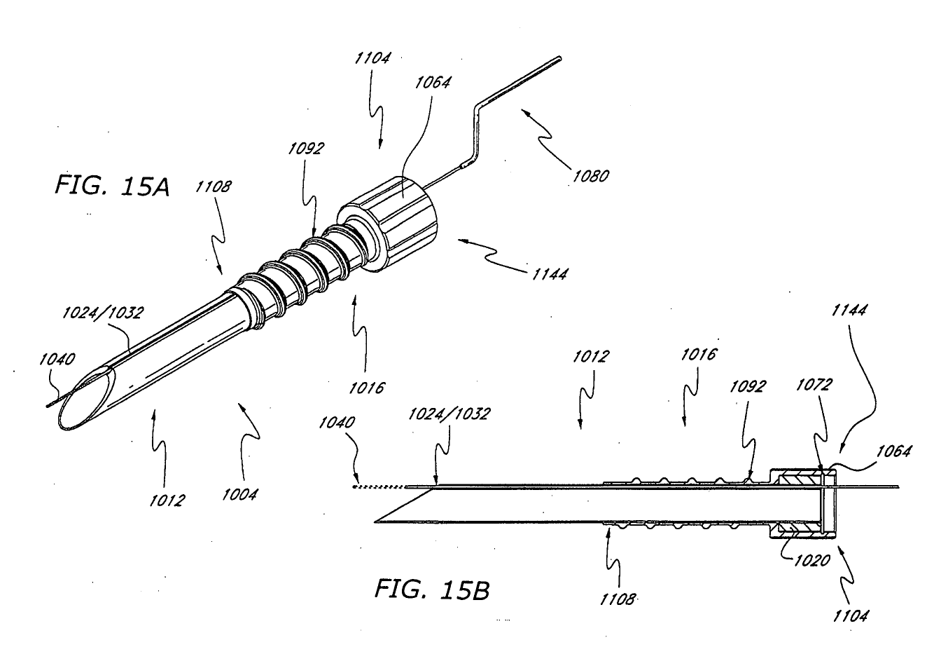

In one embodiment, there is provided a tensioning handle 1016 configured with tissue engaging threads 1092, as shown in FIGS. 15-17. As one example, FIGS. 15A-15B depict tensioning handle 1016 carried concentrically over the proximal end 1144 of an exchange cannula sub-assembly 1004 with a 30 degree eccentric exchange cannula tube 1012, 30 degree hypo tube 1032, drill wire 1040, shoulder 1020, and retaining ring 1072. Tensioning handle 1016 can also be provided with exchange cannula assemblies 1000 (generic; shown in FIGS. 10A & 10B) incorporating different embodiments of exchange cannula tubes 1012, including a 45 degree exchange cannula tube 1012.

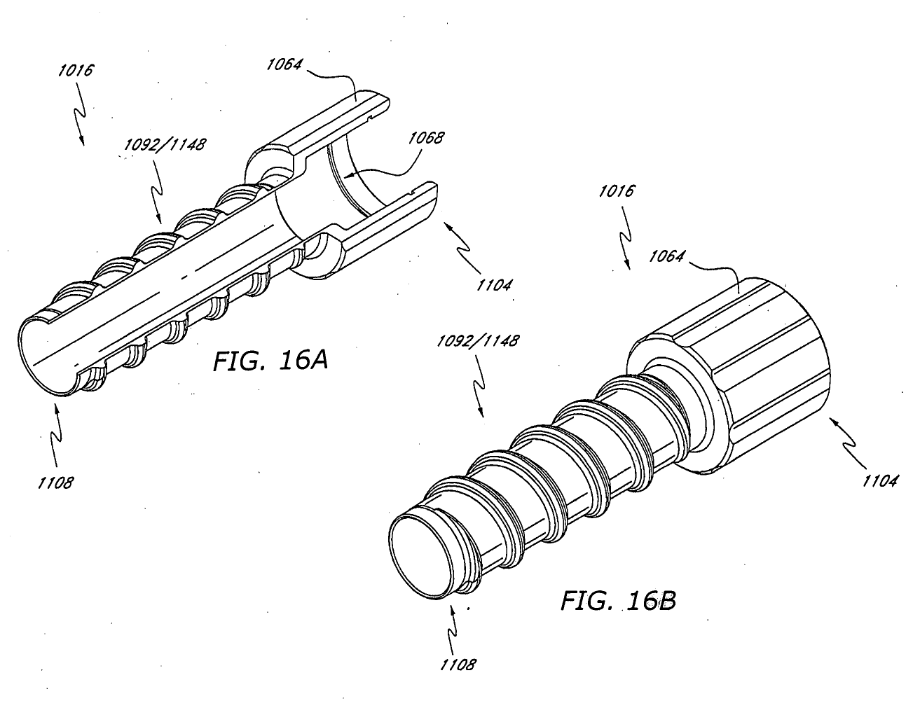

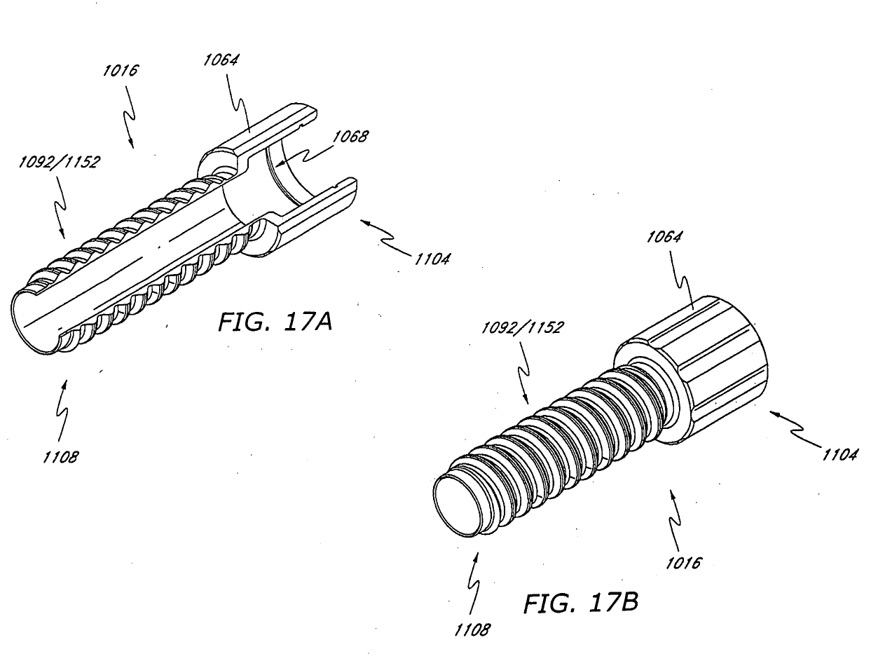

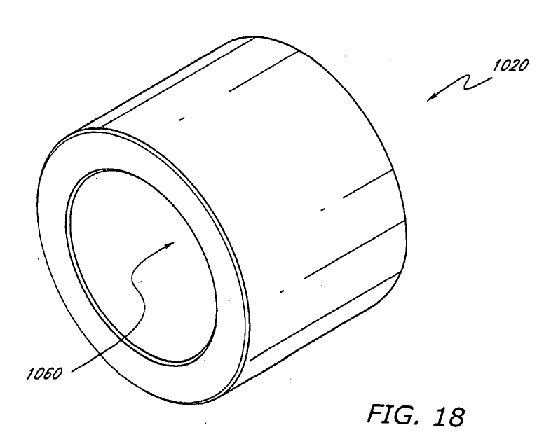

The configuration of handle 1016 facilitates distal advancement with rotation of exchange cannula assembly 1000 by the surgeon through soft tissue such that the elastic recoil of the tissue is overcome. This configuration also facilitates retention of exchange system assembly 1000, holding it in compression against the sacrum. In one embodiment, threads 1092 start at the distal end 1108 of tensioning handle 1016 and extend at least about ¾ of its length, with the remaining length comprised of a knob 1064 on its proximal end 1104. Knob 1064 can be configured with an interior slot 1068, shown in FIGS. 16A and 17A, to engage retaining means 1072, shown in FIG. 15B. Retaining means 1072 acts as a mechanical stop to distal, axial translation of tensioning handle 1016 with respect to the exchange cannula 1012. In one embodiment, shown in FIG. 15B, retaining means 1072 comprises a compressible metal snap C ring, such as Stainless Steel Internal Retaining Ring for 1″ (25.4 mm) Bore Diameter, product # 91580A211 from McMaster-Carr, http://www.mcmaster.com.

As shown in FIGS. 16A-16B and 17A-17B, tensioning handle 1016 is an elongate tube extending between a distal end 1108 and a proximal end 1104, and can range between about 3″ (75 mm) to about 6″ (155 mm) in overall length, and is often about 4″ (100 min). The outer diameter of tensioning handle 1016 ranges from between about 0.800″ (20 mm) and about 1″ (25 mm) and is often about 0.900.″ (22 mm) The inner diameter of tensioning handle 1016 ranges from between about 0.650″ (16 mm) and about 0.800″ (20 mm) and is often about 0.690″ (17 mm). The major and minor thread diameters of the threads 1092 of tensioning handle 1016 range from between about 0.710″ (18 mm) and about 0.860″ (22 mm) and about 0.860″ (22 mm) and about 1.010″ (26 mm) respectively.

In one embodiment of the invention, threaded tensioning handle 1016 is configured (e.g., molded) from a polymeric material, and the threads are not so sharp as to tear or otherwise compromise soft tissue when torque is applied to threaded tensioning handle 1016 to axially and distally advance exchange cannula sub-assembly 1004 through such soft tissue and into the sacrum. In one embodiment, there is provided a coarse-pitch threaded tensioning handle 1016, as shown in FIGS. 16A-16B, wherein coarse-pitch threads 1148 range between about 1 to about 4 threads per inch (tpi), and often between about 2 to 4 tpi. FIG. 16A depicts a cross-section of a coarse-pitch threaded tensioning handle 1016. FIG. 16B depicts a perspective view of a coarse-pitch threaded tensioning handle 1016. In yet another embodiment, there is provided a fine-pitch threaded tensioning handle 1016, as shown in FIGS. 17A-17B, wherein fine-pitch threads 1152 are greater than 4 threads per inch (tpi), and range up to about 10 tpi, with threads 1152 often between about 6 to 8 tpi. FIG. 17A depicts a cross-section of a fine-pitch threaded tensioning handle 1016. FIG. 17B depicts a perspective view of a fine-pitch threaded tensioning handle 1016.

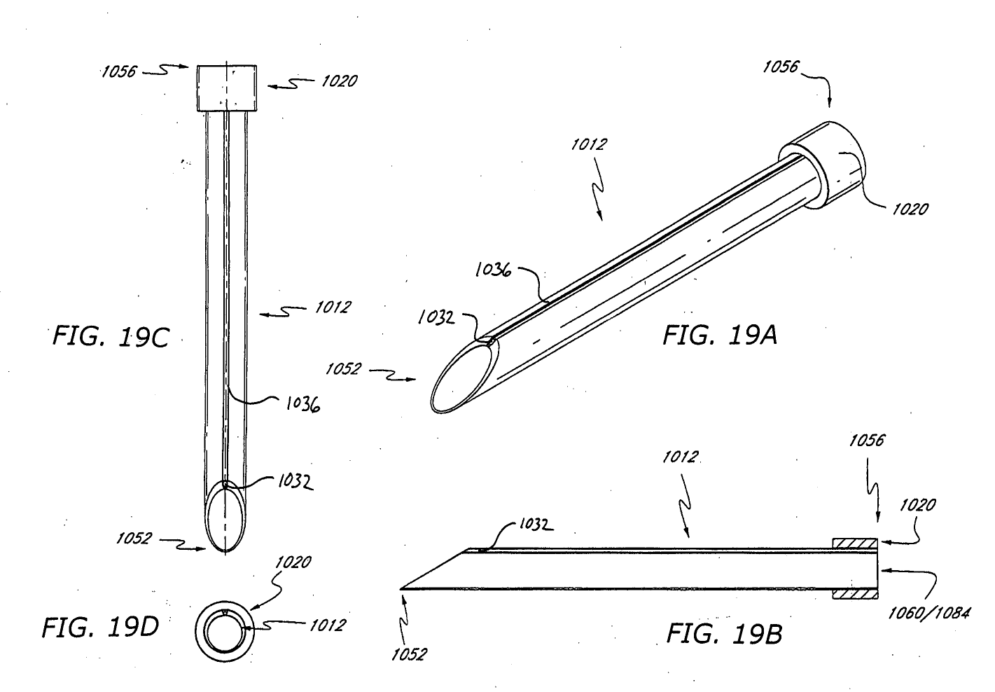

In one aspect of the present invention, exchange cannula sub-assembly 1004 comprises a shoulder 1020 shown in FIG. 18 which is placed over proximal end 1056 of exchange cannula tube 1012. Shoulder 1020 comprises a central lumen 1060 and can be press fit and brazed at assembly while maintaining the outer diameter of exchange cannula tube 1012. FIGS. 19A-19D, as one example, show shoulder 1020 press fit and brazed or otherwise secured at assembly over the proximal end 1056 of a 30 degree eccentric exchange cannula tube 1012. Tensioning handle 1016 may therefore be advanced over the distal end 1052 and advanced proximally until it is seated over shoulder 1020 and extends distally beyond shoulder 1020. Shoulder 1020 acts as a mechanical stop to the axial advance of tensioning handle 1016 in the proximal direction but does not prevent handle 1016 from rotating freely around exchange cannula tube 1012.

Shoulder 1020 can be configured from a polymeric material such as Delrin™ acetal polymer, obtained from DuPont Company, Wilmington Del., or other machinable or extrudable polymers or copolymer materials, or combinations thereof, that will withstand multiple sterilization cycles (including but not limited to) suitable materials such as, for example, polyethylene (including both high and low density polyethylenes, as appropriate ); PEEK; polycarbonate; acrylic polymers; nylon; polypropylene; PVC; ABS or other acetal copolymers, or metal or metal alloy, and is typically formed, e.g., extruded or machined, as a cylinder comprising central lumen 1060 whose diameter fits closely over the outer diameter of exchange cannula tube 1012. The diameter of central lumen 1060 ranges from between about 0.600″ (15 mm) and about 0.750″ (19 mm) and is often about 0.685″ (17 mm). The outer diameter of shoulder 1020 ranges from between about 0.900″ (22 mm) and about 1.00″ (26 mm) and is often about 0.990″ (25 mm). The axial length of shoulder 1020 ranges from between about 0.50″ (12 mm) and about 1.00″ (26 mm) and is often about 0.750″ (19 mm)

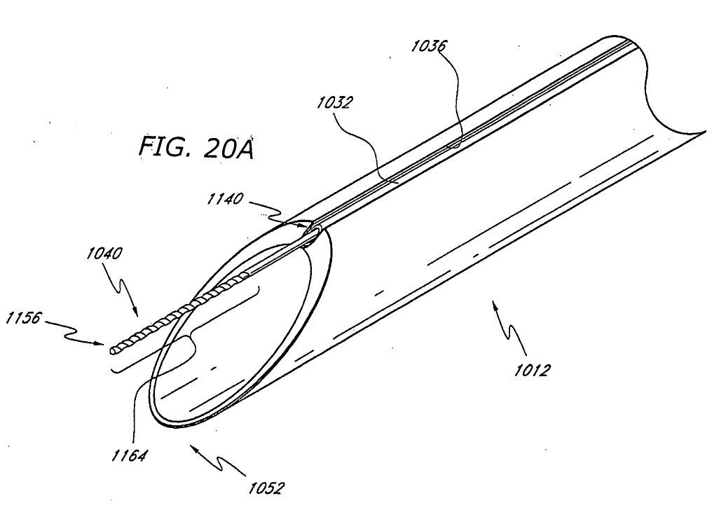

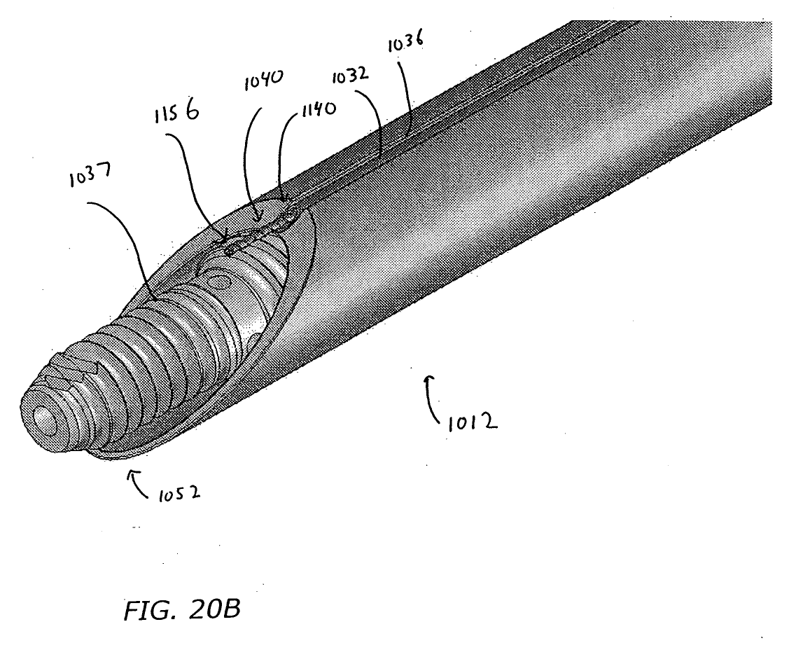

With reference to FIG. 20A, in one aspect of the present invention, exchange cannula sub-assembly 1004 comprises an elongated drill wire 1040 which extends between a distal end 1156 and a proximal end 1160 (not shown) and that is preferably formed as a solid rod from a metal alloy, e.g., 17-4 stainless steel or other suitable materials that (dimensionally) meet the ability to be torqued into the sacrum. Drill wire 1040 extends proximally beyond proximal end 1056 (not shown) of exchange cannula tube 1012. Drill wire 1040 can also extend distally beyond distal end 1052 of exchange cannula tube 1012. For example, as shown in FIG. 20A, drill wire 1040 resides in a hypo tube 1032 placed in tract 1036 and its distal end 1156 extends beyond distal end 1140 of hypo tube 1032 and distal end 1052 of exchange cannula tube 1012. FIG. 20B, in turn, illustrates the drill wire 1040 and exchange tube 1012 of FIG. 20A with a fusion implant 1037 extending partially past the distal end 1052 ofthe tube 1012.

In one embodiment, drill wire 1040 is between about 6″ (150 mm) and about 16″ (400 mm) in overall length, and is often about 14″ (355 mm). The outer diameter of drill wire 1040 is, in some embodiments, about 0.047″ (1.2 mm) and is sized to permit insertion through a portal 1024, such as hypo tube 1032, along dorsal surface 1136 of exchange tube 1012. Distal end 1156 of drill wire 1040 may be provided with a drilling or bone engaging structure such as tip 1076, as described below. One or two or more drill wires may be provided, depending upon the desired extent of anchoring.

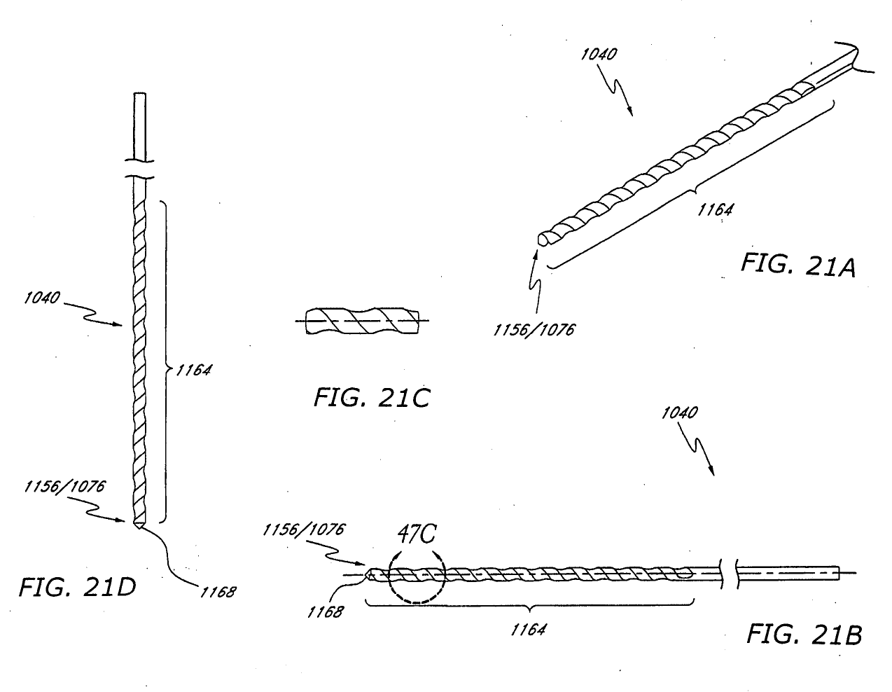

As illustrated in FIGS. 20A, 21A-D, and 22A, in one embodiment, at least a distal portion 1164 of drill wire 1040 is fluted over its length. Distal portion 1164 can range in length from about 0.2″ (5 mm) to about 8″ (205 mm) and is often about 1″ (25 mm). FIG. 21C shows an enlarged view of a segment of fluted distal portion 1164. The axial distance between the beginning and end of one flute can range from about 0.020″ (0.5 mm) to about 0.200″ (5 mm) and is often about 0.090″ (2.25 mm). Distal end 1156 of distal portion 1164 can be formed into, e.g., a conical tip 1076 as shown in FIGS. 21B and 21D with the angle between the slant 1168 of the tip and a longitudinal axis of drill wire 1040 ranging from about 15 degrees to about 60 degrees and often being about 45 degrees.

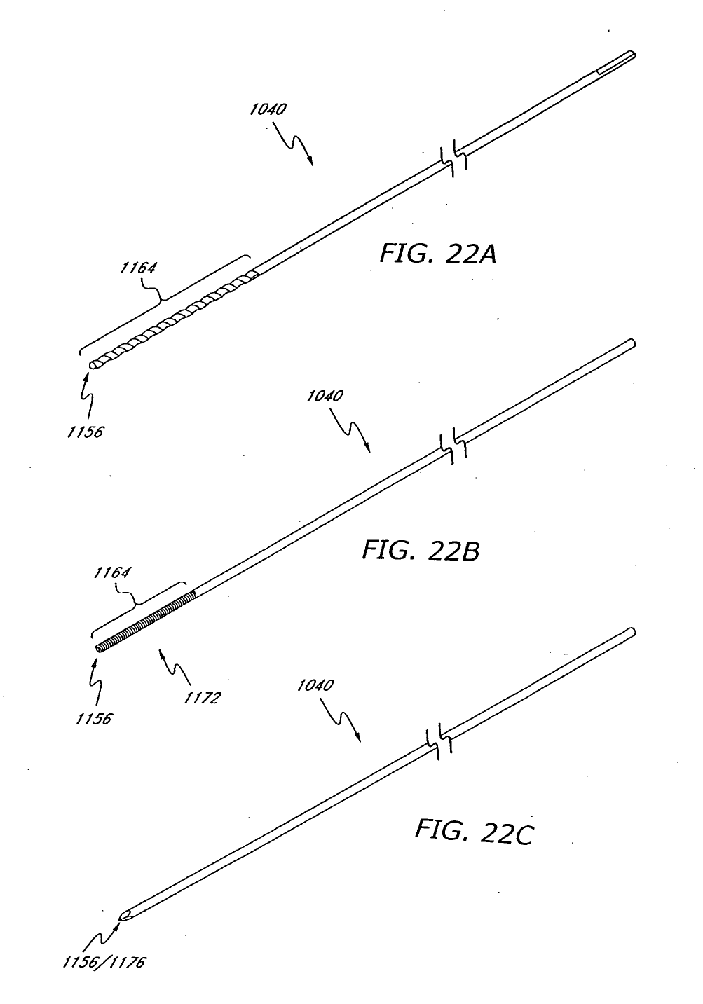

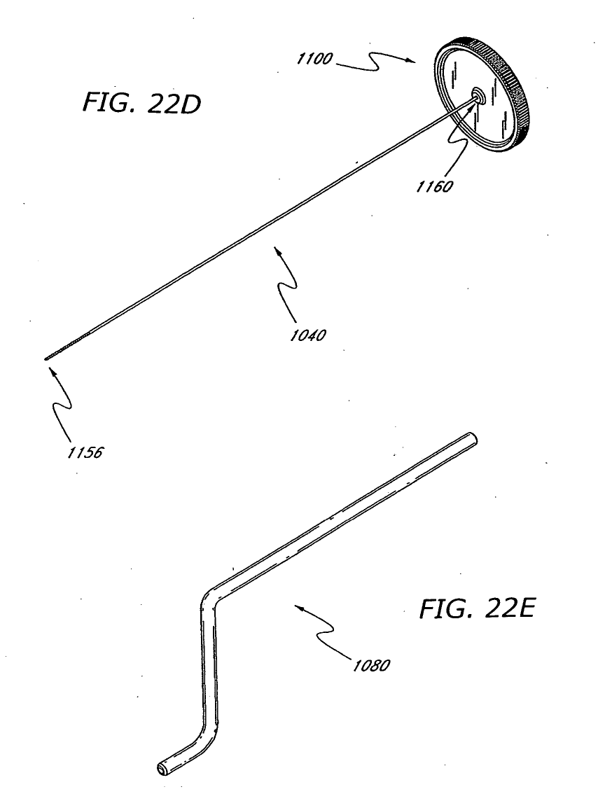

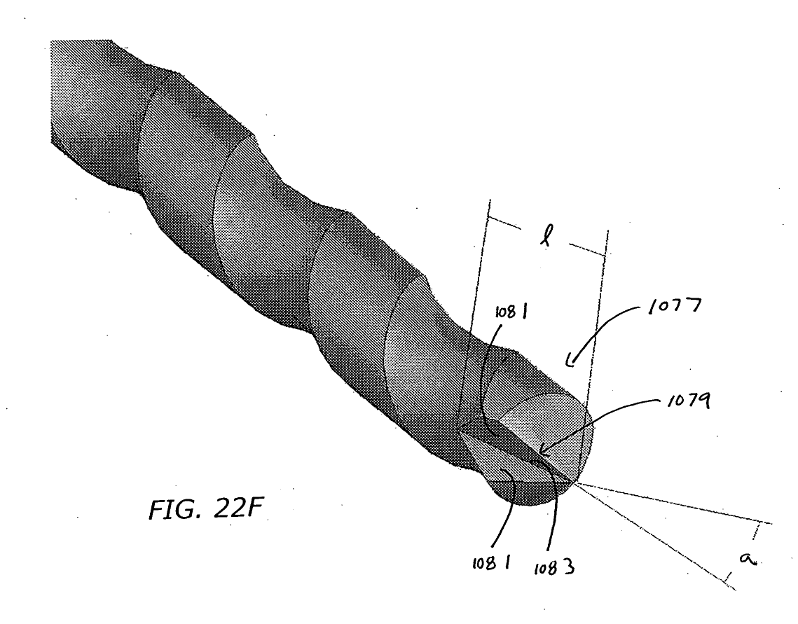

FIG. 22F illustrates an alternative drill wire configuration in which the distal tip 1077 comprises a single “gashed” flute 1079. With reference to the illustrated embodiment of FIG. 22F, the flute 1079 can be formed by the intersection of two substantially planar faces or planes 1081, which form a corner 1083. The length l of the flute 1079 can range from between about 0.060″ (1.5 mm) to about 0.250″ (6 mm), and can often be about 0.150″ (4 mm). The angle α of the flute 1079 relative to the longitudinal axis of the wire 1040 can range from between about 15 degrees to about 60 degrees and can often be about 30 degrees. The angle between the two planes 1081 of the flute 1079 can range from between about 30 degrees to about 120 degrees, and can often be about 90 degrees.

In yet another aspect of the invention, as shown in FIG. 22B, distal portion 1164 of drill wire 1040 is threaded or configured with, e.g., rolled threads 1172 over its length wherein axial advance in the distal direction is more by means of screwing than coring. In one aspect of the present invention, distal portion 1164 of drill wire 1040 comprises a tip 1076 that may be shaped into various configurations. In one embodiment, drill wire tip 1076 is formed as a simple conical or two sided wedge pointed tip (not shown). In yet another embodiment, tip 1076 is formed as a trocar tip 1176 as shown in FIG. 22C that has a three-sided bevel at, for example, 15 degrees. In still another embodiment (not shown), tip 1076 is formed as a beveled tip that has one side beveled at an angle (not shown). Often, the angle can range from between about 30 degrees to about 60 degrees relative to the longitudinal axis of drill wire 1040. The selection of the drill wire configuration and tip geometry is influenced by performance in terms of securing exchange cannula sub-assembly 1004 to the anatomy of the patient's sacrum, and cost factors.

In another aspect of the present invention, as depicted in FIGS. 22D and 22E, respectively, a handle 1100 such as a knurled knob or thumb wheel, or crank handle 1080 at the proximal end 1160 of drill wire 1040 facilitates manipulation of drill wire 1040 by the surgeon, e.g., axial advance of drill wire 1040 in the distal direction into the sacrum via tapping or application of torque.

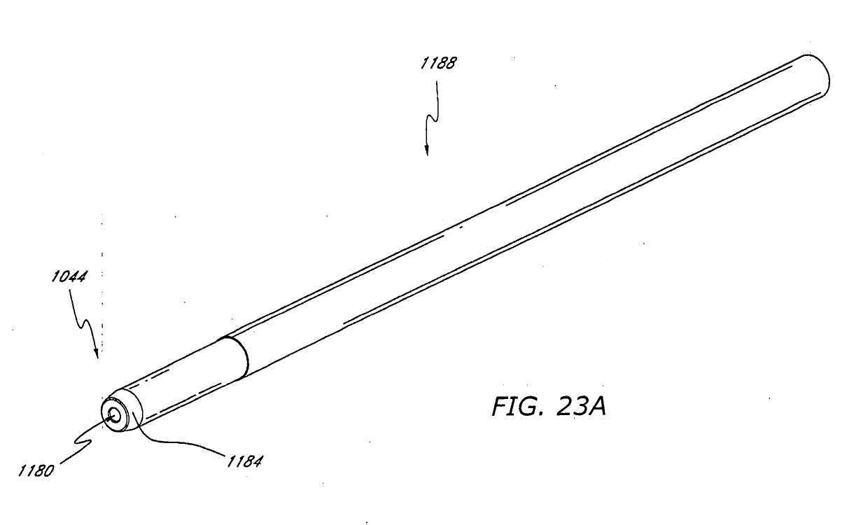

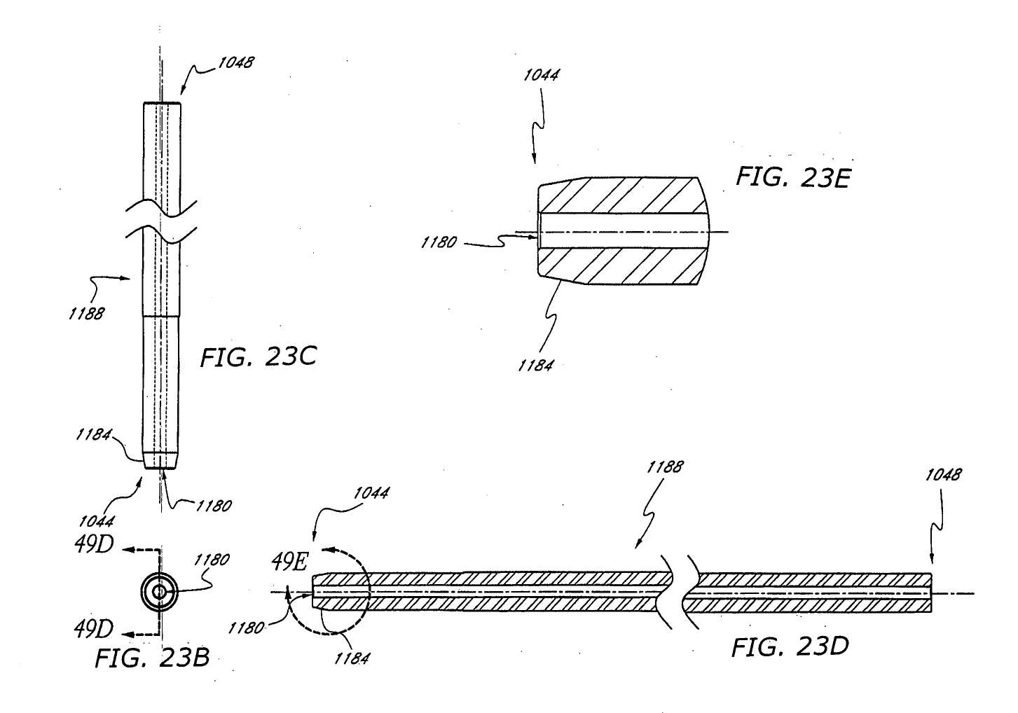

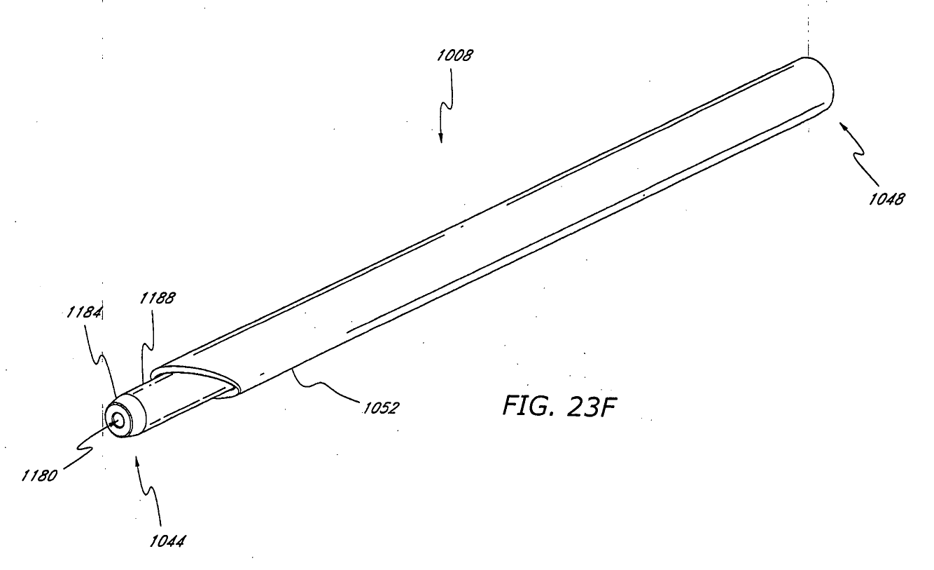

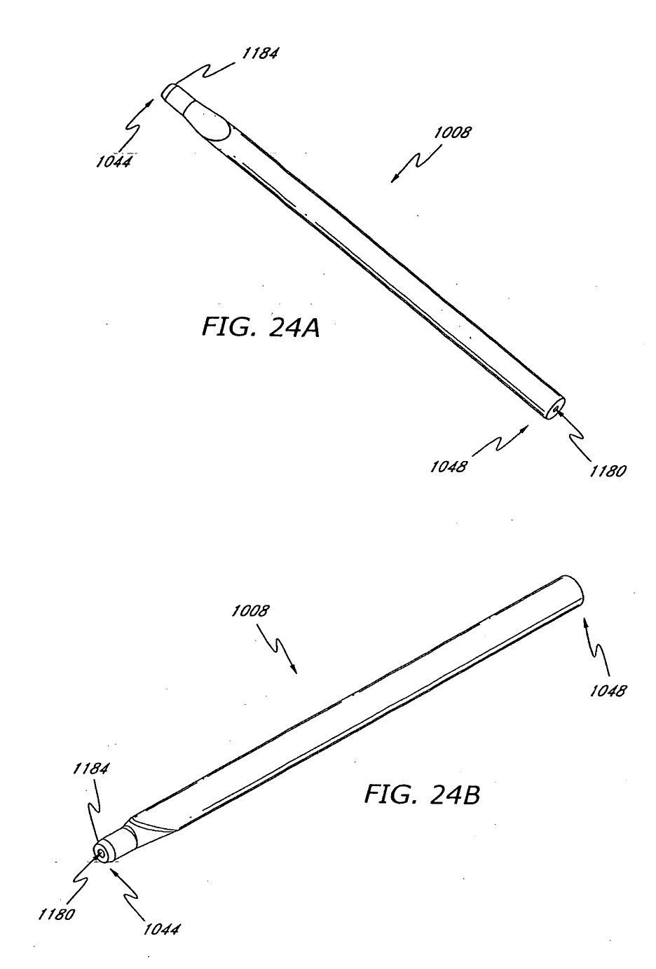

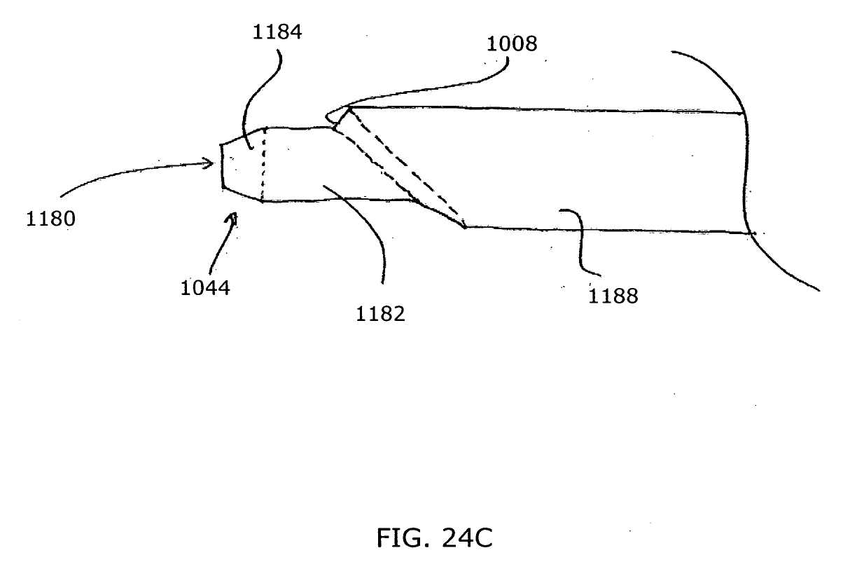

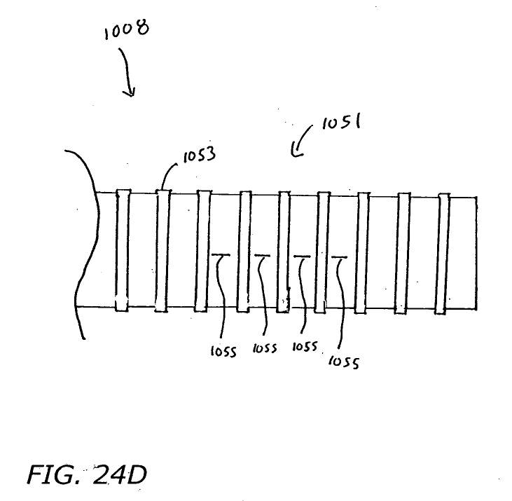

As shown in FIGS. 23F and 24A-24D, exchange bushing or rod 1008 extends between a distal end 1044 and a proximal end 1048. Exchange rod 1008 is generally elongated in shape and tapered or stepped towards a reduced diameter at distal end 1044. In a preferred embodiment, exchange rod 1008 has a hydrophilic coating and is cannulated with a central lumen 1180 (i.e., along its longitudinal axis) with a diameter ranging from about 0.10″ (2.5 mm) to about 0.25″ (6.5 mm) so as to accommodate a guide pin. For example, in one preferred embodiment, central lumen 1180 has a diameter of about 0.14″ (3.5 mm), i.e., slightly larger than the diameter of a typical guide pin, over which it is inserted. The outer diameter of exchange rod 1008 can range from between about 0.375″ (9 mm) to about 0.625″ (16 mm), and is often about 0.410″ (10 mm) with a tapered distal end 1044 increasing to about 0.560″ (14 mm) at proximal end 1048. Exchange rod 1008 may be machined, extruded, or injection molded. The length of exchange rod 1008 is generally between about 30% to about 50% longer in length than exchange cannula 1012, often approximately between about 10.00″ (250 mm) and about 14.00″ (355 mm). In one embodiment, as shown in FIGS. 23A-23F and 24A-24D, exchange rod 1008 has a tapered tip 1184 at distal end 1044 with an outer diameter of about 0.375″ (9 mm)

Delivery of exchange rod 1008 can be performed over an extended guide pin, for an atraumatic introduction through soft tissue, through which exchange cannula sub-assembly 1004 is subsequently advanced into its proper target location. In one embodiment, illustrated in FIG. 23F, exchange rod 1008 is formed of two parts, an insert 1188 and a tubular sheath 1052 with insert 1188 affixed to sheath 1052. FIGS. 23A-23E depict insert 1188 and FIG. 23F depicts an assembled exchanged rod 1008 comprising insert 1188 and sheath 1052. Insert 1188 may be affixed to sheath 1052 using any appropriate method as would be known to one skilled in the art, including, for example, welding, crimping, threadable or other interference engagement.

Additional details of an exchange rod 1008 can be seen with reference to FIG. 24C. The distal end 1044 of exchange rod 1008 is provided with a leading segment 1182, positioned between a distal tapered tip 1184 and a proximal taper 1186. The leading segment 1182 is dimensioned to fit within the sacral bore, for anchoring the exchange rod 1008 with respect to the bone. The distally tapered tip 1184 facilitates entry into the bone bore, and the proximal taper 1186 facilitates seating the exchange rod 1008 against the surface of the sacrum, at the predetermined access angle. In the illustrated embodiment, the proximal taper 1186 is inclined with respect to a longitudinal axis of the exchange rod 1008 at an angle of about 45°. Due to patient to patient anatomical variations exchange rods with any of a variety of angles may be provided for proximal taper 1186. In general, the angles will be normally within the range of from about 20° to about 60°, and specific embodiments at an angle of about 30° and at an angle of about 45° are contemplated.

The leading segment 1182 has an outside diameter adapted to cooperate with the desired sacral bore diameter. In general, this will be within the range of from about 0.25″ (6 mm) to about 0.5″ (13 mm). In one embodiment, the outside diameter leading segment 1.182 is about 0.35″ (9 mm). The body 1188 proximally of the proximal taper 1186 has an outside diameter that is coordinated to cooperate with other instruments in the procedure as has been discussed. In general, the outside diameter of the body 1188 proximally of the proximal taper 1186 will be within a range of from about 0.4″ (10 mm) to about 0.8″ (20 mm). In one embodiment, the outside diameter is approximately 0.56″ (14 mm).

The axial length of the leading segment 1182 may also be varied, with the short length excluding the distally tapered tip 1184 and the proximal taper 1186 of at least about 0.2″ (5 mm) and generally within the range of from about 0.2″ (5 mm) to about 0.6″ (16 mm) presently contemplated.

The proximal taper 1186 is designed to cooperate with a surface of the sacrum, to provide a firm seat for the exchange rod 1008. As a consequence, the clinician is preferably enabled to determine the rotational orientation of the distal end of the exchange rod 1008 from the proximal end of the exchange rod 1008. For this purpose, the proximal end of the exchange rod 1008 is preferably provided with an indicium of the rotational orientation of the distal end. One or two or more markings or other indicators on the proximal end of the body 1188 such as by laser etching, paint, engraving or otherwise, may be utilized to indicate, for example, rotational alignment with the distal most or proximal most aspect of the proximal taper 1186. Accordingly, the rotation orientation of the bevel can be maintained once the instrument is within the body cavity and often not reliably visible fluoroscopically. For example, FIG. 24D illustrates an embodiment of a proximal end or handle 1051 of the exchange rod 1008. The handle 1051 can extend proximally behind the proximal end 1056 of the cannula annular 1012 and can include grip features, such as, for example, knurling, bumps, and/or grooves and ridges 1053 (as shown in the illustrated embodiment) for aiding manipulation of the exchange rod 1008. The handle 1051 can also include indicium 1055 (e.g., laser marks, bumps, arrows, etc.) to indicate, for example, the rotational alignment of the rod 1008 as described above.

As mentioned above, the exchange bushing or rod 1008 can be fabricated as a polymeric overmold on a preformed metal bushing subcomponent. The addition of an overmolded polymeric tip to produce a polymer-metal hybrid bushing, can provide manufacturing advantages in that producing the distal taper of the exchange bushing (rod) 1008 in this manner can be more cost effective and provide more design flexibility than machining the entire component from metal. For example, a mold can be developed to mold the polymeric tapered and beveled tip (see e.g., FIG. 24C), which would be over molded onto a pre-existing stainless piece of bushing stock. This hybrid configuration can be an alternative to an all stainless or all polymeric construct. Any of a variety of medical grade polymers may be used for the overmolded tip, such as, for example, polysulphones, polyphenylsulphones, PEEK, and others known in the art.

More specifically, in one embodiment, the exchange rod 1008 can be formed from a metallic core having a proximal section with an outside diameter that is substantially equal to the desired outside diameter on the finished rod. The core can also include a distal section that steps down to a smaller diameter than the desired outer diameter of the distal section of the finished exchange rod to accommodate the thickness of the overmolded material. The distal section can be then overmolded to the desired distal configuration to produce the rod 1008.

Underneath the overmolded material of the overmolded tip, the core can have a variety of structures configured to promote engagement with the overmolded material.. For example, in one embodiment, the outer surface of the core includes surface texturing, apertures, threads, circumferential ribs and/or knurling that is configured to prevent or reduce translation and/or rotation of the overmolded material relative to the core and enhance the mechanical strength of the bond between the overmolded material and the core.

The reduced diameter distal section of the core can comprise an axially extending, smaller, distal extension, extending all or nearly all the way to the distal end of the finished bushing. In such an embodiment, the core can be configured to define the central lumen the entire length of the rod. In a modified embodiment the distal section of the metal core can terminate at a point proximal to the distal end of the finished rod. In one configuration, the smaller diameter distal portion of the core can be relatively short while providing enough length to provide sufficient mechanical bonding with the overmold material. As mentioned above, in either configuration, the outer surface of the distal portion of the core can be provided with surface texturing, apertures, threads, etc. to enhance the mechanical strength of the bond. In certain embodiments, the length of the reduced diameter extension of the core can be from between about 50% to about 100% of the length of the overmolded tip. For any portion of the overmolded tip that is not supported on the inner diameter by the core, a removable core pin can be used to preserve the inner diameter of the tip during the molding process. The removable core can then be removed after the molding process to leave an appropriately sized lumen that is aligned with the lumen in the core. Advantageously, the removable core pin is configured to provide a smooth transition between the sidewalls of the lumens in the core and the overmolded tip to prevent a wire from snagging against the sidewalls.

The metallic core can also include handle stock at the proximal end of the core for forming the handle of the rod 1008 (see e.g., FIG. 24D).

A two-level exchange bushing may be configured similarly to the one level exchange bushing described above. In one embodiment, the two-level exchange bushing has an overall length of about 11.25″ (285 mm), while the one-level exchange bushing has an overall length of about 12.5″ (318 mm). The two-level exchange bushing may have a leading segment 1182 with an outside diameter of about 0.388″ (9 mm), and a body 1188 with an outside diameter of about 0.625″ (16 mm). The proximal taper 1186 on the two level exchange bushing may also be provided at a variety of angular relationships with respect to the longitudinal axis, such as at 45°, or at 30°. The short axial length side of the leading segment 1182, excluding the axial length of the distally tapered tip 1184 and proximal taper 1186, may be at least about 0.4″ (10 mm) and, in one embodiment, at least about 0.6″ (15 mm)

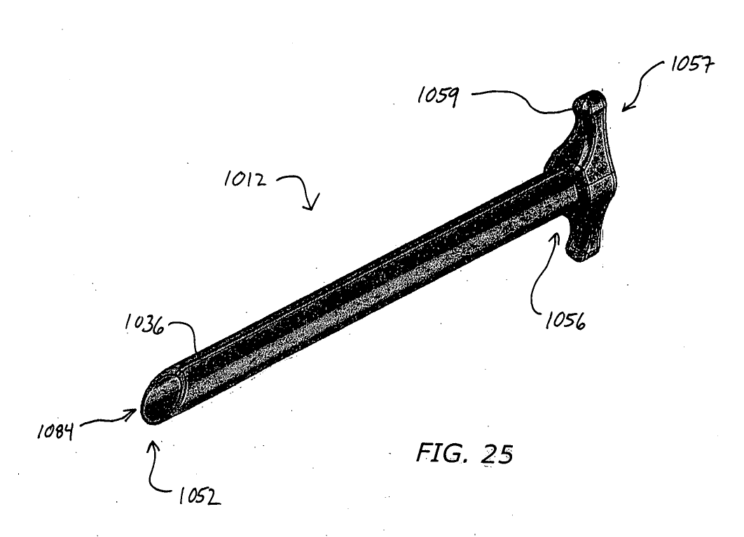

Referring to FIG. 25, there is illustrated a 45° eccentric exchange cannula sub assembly, similar to that illustrated, for example, in FIGS. 19A-D. The exchange cannula 1012 extends between the proximal end 1056 and a beveled distal end 1052. The length of the exchange cannula 1012 may be varied depending upon other instrumentation to be used in the procedure, and is generally within the range of from about 6″ (150 mm) to about 12″ (305 mm). In one embodiment of a single level exchange cannula 1012, the overall length is about 9.5″ (240 mm). In a two-level exchange cannula, the overall length is about 8″ (200 mm).

The distal end 1052 of the exchange cannula 1012 is beveled, at an angle that corresponds to the anticipated access axis with respect to the surface of the sacrum. For example, beveled angles of approximately 45° and approximately 30° with respect to the longitudinal axis of the exchange cannula 1012 are presently contemplated.

To maximize the inside diameter of the central lumen 1084 of the exchange cannula 1012 with respect to the outside diameter of the exchange cannula 1012, and accommodate the guide tract 1036, the wall thickness of the cannula 1012 tapers from the thickest portion in alignment with the tract 1036 to a thinnest portion approximately 180° away from the track 1036 as has been discussed. Track 1036 may be provided with a hypo tube 1032, as has been discussed.

The proximal end 1056 of exchange cannula 1012 is provided with an eccentric tube handle 1057. The handle 1057 may be provided in any of a variety of configurations, such as a knurled knob, pistol grip, or, as illustrated in FIG. 25, a “T” configuration. The exact shape and dimensions of the “T” handle are configured for facilitating a comfortable, firm grip by the clinician. In general, the maximum transverse dimension of the “T” handle will preferably be greater than about 1.5″ (38 mm), and, in one embodiment, is approximately 3″ (76 mm). The “T” handle may be manufactured in any of a variety of ways, such as by injection molding, machining, or other techniques known in the art.

In the illustrated preferred embodiment, the longitudinal axis of the “T” handle 1057 extends transversally to the longitudinal axis of the exchange cannula 1012. In addition, the “T” handle is rotationally oriented such that one leg of the “T” is rotationally aligned with the track 1036. A visual indicium 1059 such as a bump, indent, line, or other marker is provided on the “T” handle, so that the clinician can determine from the proximal end of the instrument the rotational orientation of the track 1036 as well as the bevel angle on the distal end 1052 of the exchange cannula 1012.

In a modified embodiment, the visual indicium 1059 on the handle 1057 of the exchange cannula 1012 is also configured to cooperate with visual indicium 1055 (see e.g., FIG. 24D) on the handle 1051 of the exchange bushing 1008. In such an embodiment, one end of the “T” handle can be provided with bump, indent, line or other marker (not shown) that is aligned with the track 1036 and bevel angle of the exchange cannula 1012 as described above. In turn, the indicium 1055 on the proximal end 1048 of the exchange bushing 1008 can be in rotational alignment with the distal most or proximal most aspect of the proximal taper 1186 (see e.g., FIG. 24C). In this manner, the distal proximal tapers 1184, 1186 of the exchange bushing 1008 can be aligned with the track 1036 and the bevel angle of the exchange cannula 1012 by aligning the indicium 1055 on the bushing 1008 with the indicium 1059 on the handle 1057 of the cannula 1012. In one embodiment, the indicium on the cannula 1012 can be disposed on a proximally facing face of the T-handle 1059.

In some embodiments, some components, such as exchange rod 1008, exchange cannula tube 1012, and tensioning handle 1016, are comprised of a polymeric material, such as Delrin TM acetal polymer, obtained from DuPont Company, Wilmington Del., or other machinable or extrudable polymers or copolymer materials, or combinations thereof, including but not limited to, suitable materials such as, for example,—polysulfone, polyvinylidene fluoride, polyethylenes, including both high and low density polyethylenes, as appropriate, PEEK, polycarbonate, acrylic polymers, nylon, polypropylene, PVC, ABS or other acetal copolymers.

In a preferred embodiment, exchange rod 1008 and exchange cannula tube 1012 are coated with a surfactant or hydrophilic coating (e.g., hydrogel) to facilitate passage through and insertion into tissue.

In another aspect of the present invention, exchange rod 1008, exchange cannula tube 1012, and other exchange cannula sub-assembly 1004 components, including, in a preferred embodiment, drill wire 1040, can be fabricated from a metal or metal alloys, such as stainless steel with biomechanical properties suitable for their intended purpose. For example, drill wire 1040 can be fabricated from metal or metal alloy, e.g., 17-4 stainless steel, that would withstand the torque experienced when it is anchored in the bone. Other materials for forming the components such that they serve their intended purpose may be suitable as would be known to those skilled in the art. In preferred embodiments, the materials forming the components of the present invention are sterilizable and biocompatible.

As used herein, the term “biocompatible” refers to an absence of chronic inflammation response or cytotoxicity when or if physiological tissues are in contact with, or exposed to the materials and devices of the present invention, including wear debris. In other aspects of the present invention the materials comprising the components of the exchange system assemblies or sub-assemblies are visible and/or imageable, e.g., fluoroscopically, or via CT (computed tomography), or MRI (magnetic resonance imaging). Contrast media such as barium sulfate or iodine, or other materials such as stainless steels like Tantalum (Ta), and Titanium (Ti), may be employed in forming these components to modify their radiolucency or radio-opaqueness if desired, taking into account contrast, detail, and special sensitivity.

Any combinations of the exchange system components, whether reusable or disposable, described above can be packaged together, for convenience at the clinical site. For example, a basic exchange system kit may include an exchange cannula subassembly 1004 and an exchange rod 1008. The exchange cannula subassembly 1004 may include any one or combination of a tensioning handle 1016, an exchange cannula tube 1012, a drill wire 1040, or other components described above. In one kit, there is provided an exchange rod 1008 and an exchange cannula tube 1012 of the type having a “T” handle at the proximal end. A kit may include 2 or 3 or 4 or more exchange cannula tubes 1012, each having a different distal end angle as has been described. For example, in one kit, a first exchange cannula tube 1012 is provided having a distal end with a 30° angled surface and a second exchange cannula tube 1012 is provided having approximately a 45° distal surface. Similarly, kits in accordance with the present invention may include 2 or 3 or 4 or more exchange rods 1008. Each exchange rod in the kit may have a different characteristic, such as a different angle of inclination as has been discussed. In one kit, a first exchange rod 1008 has a 30° angulation and a second exchange rod 1008 has a 45° angulation as has been discussed. In addition, kits in accordance with the present invention may include 2 or 3 or 4 or more drill wires of one or more tip configurations, such as described above (e.g., FIGS. 22A-22F).

Various combinations of the tools and devices described in the co-pending patent applications previously incorporated herein by reference may also be provided in the form of kits, each with or without the exchange systems described herein, so that all of the tools desirable for performing a particular procedure will be available in a single package. Kits in accordance with the present invention may include access kits, such as for achieving percutaneous access to the sacrum, and access kits for achieving soft tissue access to the sacrum and access through the sacrum into the desired treatment zone. Kits may also be provided with the tools necessary for disc preparation. Further kits may be provided with temporary distraction and/or insertion tools for insertion of implants.

Access kits may include all or any sub-combination of the following components, which have been described previously herein: one or more guide pin introducers, stylet, guide pin, guide pin handle, and guide pin extension. Each of these components may be either reusable or disposable. The access kit may additionally include one or more dilators, such as a 6 mm dilator and 8 mm dilator, and a 10 mm dilator with sheath. In one implementation of the kit, each of the dilators is reusable, and the sheath is disposable. The access kit may additionally include twist drills, such as a 6 mm, 7.5 mm and 9 mm drills which may be reusable.

Disc preparation kits may differ, depending upon whether the procedure is intended to be one level or multi-level. The disc preparation kit may include a plurality of cutters. In a single level kit, anywhere from 3 to 7 cutters and, in one embodiment, 5 cutters are provided. In a two level kit, anywhere from 5 to 14 cutters may be provided, and, in one embodiment, 10 cutters are provided. All of the cutters may be one time use disposable.

The disc preparation kit may additionally include one or more tissue extraction tools, for removing fragments of the nucleus. In a one level kit, 3 to 8 tissue extraction tools, and, in one embodiment, 6 tissue extraction tools are provided. In a two level disc preparation kit, anywhere from about to 8 to about 14 tissue extraction tools, and, in one embodiment, 12 tissue extraction tools are provided. The tissue extraction tools may be disposable.

The disc preparation kit may additionally include a bone graft inserter, which may be disposable.

An allograft kit may be provided including, in addition to the tools in the access and disc preparation kits, an allograft inserter tool and a temporary distraction tool. A selection of twist drills may be provided, such as a 9.5 mm, 10 mm, 10.5 mm, 11 mm or 11.5 mm twist drill, depending upon the size of the desired graft. The allograft kit may additionally include an exchange system, including a cannula and bushing, as have been described previously herein.

A fusion kit intended for a one level fusion may include, in addition to the tools in the access and disc preparation with bone graft inserter kits a one piece fusion rod, a rod driver, and a paste inserter. The fusion kit may additionally include a plug, a plug driver, and one or more twist drills such as a 7.5 mm and a 6 mm. The fusion kit will additionally include an exchange system as has been discussed. The rod driver and twist drills may be reusable.

In an alternate fusion kit, intended for two-level fusion, the kit may include one, two-pieces fusion rods, or one, one- piece fusion rod and one mobility implant, or a two-piece implant, one of which is a fusion implant and one of which is a mobility device The fusion kit additionally includes a rod driver, a paste inserter, one proximal and one distal plugs and two plug drivers. The fusion kit may additionally include one or more twist drills, such as a 7.5 mm and a 6 mm twist drill. The fusion kit will additionally include an exchange system.

In accordance with a further aspect of the present invention, there is provided a method of advancing a device along an axially oriented access tract. The device may, for example, be an implant, such as a spinal fusion implant or a spinal motion preservation implant, or a site preparation tool. In some embodiments, the device has a cross-sectional area greater than the inside cross-sectional area of an undilated access tract, the dilator sheath or working cannula described in co-pending and commonly assigned U.S. patent application Ser. No. 10/972,065, filed on Oct. 22, 2004, hereby incorporated by reference in its entirety. The method comprises the steps of positioning a dilator sheath between an access site on a skin surface and a target site on a bone. For example, the target site may be on the sacrum. The treatment site may be an intervertebral disc upon which a procedure is to be performed, as will be understood by one skilled in the art. Exchange rod 1008 is advanced over an extended guide pin, following removal of the dilator sheath. Exchange cannula sub-assembly 1004 is axially and distally advanced through soft tissue, by means of applying torque to tensioning handle 1016 which de-tensions the natural spring action of the soft tissue, and over exchange bushing 1008. More specifically, distal end 1052 of exchange cannula tube 1012 is slipped into the access tract over exchange rod 1008 and is seated against the sacrum. Exchange cannula sub-assembly 1004 is then secured into the sacrum by means of the drill 1040 which is tapped or torqued into the sacrum to a depth of about 5 mm to about 10 mm. After the exchange cannula sub-assembly 1004 is securely anchored into the sacrum, exchange bushing 1008 is removed, leaving exchange cannula 1012 in position along the access tract. Devices, implants, or other instrumentation can then be inserted, in combination with and over a guide pin or independently, through lumen 1084 of exchange cannula tube 1012. See, e.g., FIG. 20B.

Introducing devices, implants, or other instrumentation may involve enlarging the diameter of at least a portion of exchange cannula tube 1012. The method may additionally comprise radially enlarging a portion of distal end 1052 of exchange cannula tube 1012. The access tract may have a longitudinal axis which intersects the surface of the bone at an angle, and distal end 1052 of the exchange cannula tube 1012 is beveled at an angle that corresponds to the angle at which the axis intersects the surface of the bone.

Embodiments of the present invention disclose devices that may be used to form or enlarge a posterior or anterior percutaneous tract, access, or otherwise prepare vertebral elements and inter-vertebral motion segments for fusion or dynamic stabilization via implantation of therapeutic agents and materials and spinal devices. It will be noted that the tools described can be used for and with the introduction of any number of devices, such as, for example, fusion devices, motion preservation devices, etc. Instrumentation is introduced and aligned through the percutaneous pathways and according to the trans-sacral axial access methods disclosed in U.S. patent application Ser. No. 10/972,065, filed on Oct. 22, 2004 and by Cragg, in commonly assigned U.S. Pat. Nos. 6,558,386, 6,558,390, and 6,575,979, each incorporated herein in their entirety by reference. For example, to ensure that the tract along which instrumentation is introduced is positioned as desired, fluoroscopy, endoscopy, or other radio-imaging means may be used to aid alignment.

In another aspect, the present invention provides a series of surgical tools and devices, wherein the preferred embodiments of each are configured and constructed in accordance with optimal intended function and in deference to biomechanical and safety constraints. For example, the tools and devices may be cannulated, solid, blunt, beveled, angled, retractable, fixed, tilted, axially aligned, offset, extendable, exchangeable, stiff, flexible, deformable, recoverable, anchored, removable, biocompatible, able to be sterilized & machined, moldable, reusable, or disposable.

Some of the devices disclosed herein may be used in combination or sequentially with other devices. The devices may comprise solid or hollow elongated members. In designing the devices, some design parameters, such as outer diameter, must be constrained so as to accommodate the patient anatomies that the devices will engage. For those devices that are used in combination with, over, or through other devices, design parameters such as wall thickness, mechanical strength, inner diameter, and materials used in forming the devices may also be modified so as to enable engagement of patient anatomies without incurring deformation or otherwise inhibiting functionality. Certain of these solid body and hollow body members can have distal means, mechanisms, or apertures that may be configured or manipulated for either precluding or facilitating engagement with tissue, the latter including piercing, tapping, dilating, excising, fragmenting, extracting, drilling, distracting (e.g. elevating), repairing, restoring, augmenting, tamping, anchoring, stabilizing, fixing, or fusing tissue. Certain of these solid body and hollow body members can have proximal means or mechanisms, such as pins, slots or apertures that may be configured or manipulated to engage, grasp, twist, pilot, angle, align, extend, expose, retract, drive, attach or otherwise enable or facilitate the functionality of other components within the surgical toolset, e.g., the distal means and mechanisms noted above in this paragraph.

In one preferred embodiment, devices are aligned axially, under visualization, and progressively inserted into a human lumbar-sacral spine through the minimally invasive percutaneous entry site adjacent the coccyx to access the L5-S1 or L4-L5 disc space to enable the subsequent introduction and deployment of spinal stabilization devices. The instrumentation systems and techniques disclosed herein prepare an inter-vertebral motion segment for subsequent receipt of therapeutic procedures and enable axial placement of implants close to and in alignment with the human spine's physiological center of rotation, more effectively, with less trauma, and without the residual negative physiological impacts that may occur as a result of invasion of the annulus using other systems and techniques.

Other specific advantages over current practice include the following: the patient is in a prone position which facilitates introducing other instrumentation from a posterior position, blood loss to soft tissue structures is minimal, e.g., veins, arteries, nerves are preserved, and the patient is subjected to surgery and anesthesia for substantially less time than with conventional procedures.

While the present invention has been illustrated and described with particularity in terms of preferred embodiments, it should be understood that no limitation of the scope of the invention is intended thereby. For example, features of any of the foregoing methods, and exemplary devices may be substituted or added into the others, as will be apparent to those of skill in the art. The scope of the invention is in no way intended to be limited by the brevity or exemplary nature of the material above, and may be further understood from the accompanying figures and claims.

What is claimed is:

1. An exchange system for increasing the cross sectional area of an access pathway through soft tissue to a surface of a bone, comprising:
an elongate, tubular exchange bushing, having a proximal end, a distal end and a central lumen extending therethrough; and
an elongate, tubular exchange cannula, having a proximal end, a distal end and a central lumen extending therethrough, the exchange cannula comprising a beveled distal end;
a handle on the proximal end of the exchange-cannula;
wherein the exchange bushing is axially slidable within the central lumen of the exchange cannula.

2. An exchange system as in claim 1, wherein the bevel is at an angle within the range of from about 20 degrees to about 70 degrees with respect to a longitudinal axis of the exchange cannula.

3. An exchange system as in claim 1, further comprising on the handle an indicium of the rotational orientation of the bevel.

4. An exchange system as in claim 1, wherein the exchange bushing further comprises a beveled distal end and the beveled distal end of the exchange cannula is at an angle that is substantially the same as the angle of the beveled distal end of the exchange bushing.

5. An exchange system, comprising:
an exchange rod; and
an exchange cannula sub-assembly, wherein in the exchange cannula sub-assembly further comprises:
an exchange cannula tube;
a drill wire portal;
a drill wire;
a tensioning handle;
a shoulder; and
a retainer.

6. An exchange system as in claim 5, wherein the drill wire comprised in the exchange cannula sub-assembly further comprises a drill handle.

7. An exchange system as in claim 5, wherein the exchange cannula tube comprises a proximal end, a distal end, and a central lumen.

8. An exchange system as in claim 7, wherein the distal end comprises a bevel at its distal end.

9. An exchange system as in claim 8, wherein the bevel comprises a surface slanted relative to a longitudinal axis of the exchange cannula tube at an angle between about 20 degrees and about 70 degrees.

10. An exchange system as in claim 7, wherein the exchange cannula tube has an eccentric wall thickness.

11. An exchange system as in claim 7, wherein the exchange cannula tube has a length between about 6.00″ and about 10.00″

12. An exchange system as in claim 7, wherein the exchange cannula tube has a length of about 8.00″.

13. An exchange system as in claim 7, wherein the exchange cannula tube has an inner diameter between about 0.375″ and about 0.700″.

14. An exchange system as in claim 7, wherein the exchange cannula tube has an outer diameter between about 0.650″ and about 0.800″.

15. An exchange system as in claim 5, wherein the drill wire portal comprises a groove on the dorsal surface of the exchange cannula tube aligned with a longitudinal axis of the exchange cannula tube.

16. An exchange system as in claim 15, wherein the drill wire portal further comprises a hypo tube positioned in the groove.

17. An exchange system as in claim 5, wherein the drill wire portal is configured as an integral lumen formed just below the dorsal surface of the exchange cannula tube aligned with the longitudinal axis of the exchange cannula tube.

18. An exchange system as in claim 5, wherein the drill wire portal comprises a hypo tube seated along the dorsal surface of the exchange cannula tube aligned with the longitudinal axis of the exchange cannula tube.

19. An exchange system as in claim 5, wherein the tensioning handle further comprises a knob and threads.

20. An exchange system as in claim 19, wherein the knob comprises an interior slot to engage the retainer.

21. An exchange system as in claim 19, wherein the threads are coarse-pitch.

22. An exchange system as in claim 19 wherein the threads are fine-pitch.

23. An exchange system as in claim 5, wherein the tensioning handle has a length ranging from about 3.00″ to about 6.00″.

24. An exchange system as in claim 5, wherein the tensioning handle has an outer diameter ranging from between about 0.8″ and about 1.0″.

25. An exchange system as in claim 5, wherein the tensioning handle has an inner diameter ranging from between about 0.650″ and about 0.800″.

26. An exchange system as in claim 5, wherein the shoulder has an inner diameter ranging from between about 0.600″ and about 0.750″.

27. An exchange system as in claim 5, wherein the shoulder has an outer diameter ranging from between about 0.900″ and about 1.00″.

28. An exchange system as in claim 5, wherein the shoulder has an axial length of between about 0.50″ and about 1.00″.

29. An exchange system as in claim 5, wherein the drill wire has a length between about 6.00″ and 16.00″.

30. An exchange system as in claim 5, wherein the drill wire has an outer diameter of between about 0.03″ and about 0.05″.

31. An exchange system as in claim 5, wherein the drill wire comprises a fluted portion.

32. An exchange system as in claim 5, wherein the drill wire comprises a flute disposed at a distal tip of the drill wire.

33. An exchange system as in claim 32, wherein the flute has a length between about 0.060″ (1.5 mm) and about 0.250″ (6 mm).

34. An exchange system as in claim 32, wherein the flute comprises a first substantially planar surface and a second substantially planar surface that intersect to form a corner.

35. An exchange system as in claim 34, wherein the corner extends along a line that is positioned at an angle relative to a longitudinal axis of the drill wire of between about 15 degrees and about 60 degrees.

36. An exchange system as in claim 34, wherein an angle between the first substantially planar surface and the second substantially planar surface is between about 30 degrees and about 120 degrees.

37. An exchange system as in claim 5, wherein the drill wire comprises a threaded portion.

38. An exchange system as in claim 5, wherein the drill wire comprises a trocar tip.

39. An exchange system as in claim 5, wherein the drill wire comprises a beveled tip.

40. An exchange system as in claim 5, wherein the exchange rod further comprises a distal end, a proximal end, and a central lumen.

41. An exchange system as in claim 40 wherein the exchange rod further includes a handle at the proximal end.

42. An exchange system as in claim 41, wherein the handle includes visual indicia configured to indicate rotational orientation of the exchange rod.

43. An exchange system as in claim 41, wherein the handle includes grip features configured to aid manipulation of the exchange rod.

44. An exchange system as in claim 40, wherein the central lumen has an inner diameter ranging from about 0.10″ to about 0.20″.

45. An exchange system as in claim 5, wherein the outer diameter of the exchange rod ranges from about 0.375″ to about 0.700″.

46. An exchange system as in claim 45, wherein the outer diameter of the exchange rod is about 0.410″ at the distal end and about 0.560″ at the proximal end.

47. An exchange system as in claim 45, wherein the outer diameter of the exchange rod is about 0.375″ at the distal end.

48. An exchange system as in claim 5, wherein the length of the exchange rod ranges from between about 10.00″ and about 14.00″.

49. An exchange system as in claim 5, wherein the exchange rod is formed of a single part, having a proximal section and a distal section, and the distal section has a smaller diameter than the proximal section.

50. An exchange system as in claim 5, wherein at least one of the exchange rod and the exchange cannula tube is provided with a surfactant or hydrophilic coating.

51. An exchange system as in claim 5, wherein the following components are formed of metal or metal alloys: the exchange rod, the exchange cannula tube, the drill wire, the tensioning handle, the shoulder, the retaining means, and the drill handle.

52. An exchange system as in claim 51, wherein the metal or metal alloys comprise 300 series or 17-4 stainless steel.

53. An exchange system as in claim 5, wherein the radiolucency or radio-opaqueness of one or more of the following components is modified using contrast media: the exchange rod, the exchange cannula tube, the drill wire, the shoulder, the retaining means, and the drill handle.

54. An exchange system as in claim 53, wherein the contrast media is selected from the group consisting of: barium sulfate, iodine, stainless steel, Tantalum, and Titanium.

55. An exchange system as in claim 5, wherein the exchange rod comprises a proximal end, a distal end, and a tapered transition between a small diameter distal section and a larger diameter proximal section.

56. An exchange system as in claim 55, wherein the tapered transition is inclined at an angle with respect to a longitudinal axis of the rod.

57. An exchange system as in claim 56, wherein the angle is between about 20 degrees and about 70 degrees.

58. An exchange system as in claim 56, wherein the angle is between about 30 degrees and about 60 degrees.

59. An exchange system as in claim 56, wherein the exchange rod further includes a handle at a proximal end of the exchange rod, the handle comprising visual indicia configured to indicate a rotational orientation of the tapered transition.

60. An exchange system as in claim 55, wherein at least a portion of the small diameter distal section comprises a core formed of a first material that is overmolded with a second material.

61. An exchange system as in claim 60, wherein the first material comprises a metallic material and the second material comprises a polymeric material.

62. A method of advancing a device along an access tract, comprising the steps of:
removing a dilator sheath positioned over a guide wire extending between an access site and a target site on a bone;
advancing a cannulated exchange bushing over the guide wire;
advancing a tubular exchange cannula over the exchange bushing;
removing the exchange bushing, leaving the exchange cannula in position along the access tract; and
introducing the device through the exchange cannula and through the target site to a treatment site

63. A method as in claim 62, wherein the device is an implant.

64. A method as in claim 62, wherein the device is a spinal fusion implant.

65. A method as in claim 62, wherein the device is a spinal motion preservation implant.

66. A method as in claim 62, wherein the device is a site preparation tool.

67. A method as in claim 62, wherein the device has a cross sectional area that is greater than the inside cross sectional area of the dilator sheath and the exchange cannula provides a protected portal for the surrounding tissue as the device is introduced

68. A method as in claim 62, wherein the device has a cross sectional area that is greater than the inside cross sectional area of the access track before dilation and the exchange cannula provides a protected portal for the surrounding tissue as the device is introduced.

69. A method as in claim 62, wherein the target site is on the sacrum.

70. A method as in claim 62, wherein the access tract has a longitudinal axis which intersects the surface of the bone at an angle, and the distal end of the exchange cannula is beveled at an angle that corresponds to the angle at which the axis intersects the surface of the bone.

71. A method of advancing a device, implant, or other instrumentation along an axially oriented tract, comprising the steps of:
advancing an exchange rod over an extended guide pin along the tract;
advancing an exchange cannula sub-assembly over the exchange rod;
securing the exchange cannula sub-assembly into a sacrum;
removing the exchange rod from the tract; and
inserting the device, implant, or instrumentation along the tract through a central lumen of an exchange cannula tube of the exchange cannula sub-assembly.

72. A method as in claim 71, wherein advancing the exchange cannula sub-assembly further comprises applying torque to a tensioning handle of the exchange cannula sub-assembly to advance the sub-assembly through soft tissue.

73. A method as in claim 71, wherein advancing the exchange cannula sub-assembly further comprises placing a distal end of the exchange cannula tube over the exchange rod and seating it against the sacrum.

74. A method as in claim 71, wherein securing the exchange cannula sub-assembly further comprises tapping or torquing a drill wire placed on or within the dorsal surface of the exchange cannula tube into the sacrum.

75. A method as in claim 74, wherein the drill wire is tapped or torqued a distance of between about 5 mm and about 10 mm into the sacrum.

76. A method as in claim 71, wherein prior to inserting the device, implant, or instrumentation the guide pin is removed.

77. A method as in claim 71 further comprising the step of enlarging the diameter of at least a portion of the exchange cannula tube.

78. A method as in claim 71 wherein the tract comprises a longitudinal axis which intersects the surface of the sacrum at an angle.

79. An exchange system, comprising:
an exchange cannula sub-assembly, wherein in the exchange cannula sub-assembly further comprises:
an exchange rod;
an exchange cannula tube;
a drill wire portal;
a drill wire;
a handle for the exchange cannula tube that is transverse to a longitudinal axis of the exchange cannula tube;
a shoulder; and
a retainer.
